SEQUENCE LISTING (36 sequences)

SEQ ID NO: 1 - DNA, 40 bp, Artificial Sequence
  Description of Artificial Sequence Synthetic primer
ctagataccgttcgtatagcatacattatacgaagttatg

SEQ ID NO: 2 - DNA, 40 bp, Artificial Sequence
  Description of Artificial Sequence Synthetic primer
agcttataacttcgtatagcatacattatacgaacggtag

SEQ ID NO: 3 - DNA, 47 bp, Artificial Sequence
  Description of Artificial Sequence Synthetic primer
aaacctagggccattcatgaaaaaatgttgtcctcagatgaccatcc

SEQ ID NO: 4 - DNA, 44 bp, Artificial Sequence
  Description of Artificial Sequence Synthetic primer
aaacctaggctcactcaacccccatgtccttccaactctagctg

SEQ ID NO: 5 - DNA, 42 bp, Artificial Sequence
  Description of Artificial Sequence Synthetic primer
aaagtcgacctacattggtccatcgattacattagttcttgg

SEQ ID NO: 6 - DNA, 42 bp, Artificial Sequence
  Description of Artificial Sequence Synthetic primer
aaagtcgacgaggccttgaggtcaaactccagaaccccagag

SEQ ID NO: 7 - DNA, 21 bp, Artificial Sequence
  Description of Artificial Sequence Synthetic primer
ccagaaagatggccctattga

SEQ ID NO: 8 - DNA, 22 bp, Artificial Sequence
  Description of Artificial Sequence Synthetic primer
ctccttactggggcccagtggg

SEQ ID NO: 9 - DNA, 20 bp, Artificial Sequence
  Description of Artificial Sequence Synthetic primer
caacttgcatgtggatgacc

SEQ ID NO: 10 - DNA, 21 bp, Artificial Sequence
  Description of Artificial Sequence Synthetic primer
cttgagcaggatgtgggattc

SEQ ID NO: 11 - DNA, 20 bp, Artificial Sequence
  Description of Artificial Sequence Synthetic primer
agcactggtcctatgggttg

SEQ ID NO: 12 - DNA, 20 bp, Artificial Sequence
  Description of Artificial Sequence Synthetic primer
gggccagtgcatctacatct

SEQ ID NO: 13 - DNA, 20 bp, Artificial Sequence
  Description of Artificial Sequence Synthetic primer
ccacctatgccatctccagt

SEQ ID NO: 14 - DNA, 20 bp, Artificial Sequence
  Description of Artificial Sequence Synthetic primer
accatgctgacaatgatgga

SEQ ID NO: 15 - DNA, 20 bp, Artificial Sequence
  Description of Artificial Sequence Synthetic primer
aacccagacacaagcattcc

SEQ ID NO: 16 - DNA, 20 bp, Artificial Sequence
  Description of Artificial Sequence Synthetic primer
ctgggcctggtagttgttgt

SEQ ID NO: 17 - DNA, 25 bp, Artificial Sequence
  Description of Artificial Sequence Synthetic primer
gaccccttcattgacctcaactaca

SEQ ID NO: 18 - DNA, 24 bp, Artificial Sequence
  Description of Artificial Sequence Synthetic primer
ggtcttactccttggaggccatgt

SEQ ID NO: 19 - DNA, 25 bp, Artificial Sequence
  Description of Artificial Sequence Synthetic primer
tgagaacggcatcatatttaacaac

SEQ ID NO: 20 - DNA, 22 bp, Artificial Sequence
  Description of Artificial Sequence Synthetic primer
gcccgtcagagctttcataaag

SEQ ID NO: 21 - DNA, 20 bp, Artificial Sequence
  Description of Artificial Sequence Synthetic primer
tggaggccactatccgagaa

SEQ ID NO: 22 - DNA, 22 bp, Artificial Sequence
  Description of Artificial Sequence Synthetic primer
tgttagccttgtgtgggatgag

SEQ ID NO: 23 - DNA, 23 bp, Artificial Sequence
  Description of Artificial Sequence Synthetic primer
tcatgcgaaagggaattatagga

SEQ ID NO: 24 - DNA, 25 bp, Artificial Sequence
  Description of Artificial Sequence Synthetic primer
tgcttgtagtgctcatcaaatctct

SEQ ID NO: 25 - DNA, 26 bp, Artificial Sequence
  Description of Artificial Sequence Synthetic primer
cgggatccagacgaccacaaatccag

SEQ ID NO: 26 - DNA, 29 bp, Artificial Sequence
  Description of Artificial Sequence Synthetic primer
ccaagcttgattcataactcacctgtggc

SEQ ID NO: 27 - protein, 21 aa, Mus sp.
AIHEKMLSSDDHPRRPQIQKC

SEQ ID NO: 28 - DNA, 2860 bp, Homo sapiens
actgagcaaatgagatagaaacatggcattcttaattatactaattacctgctttgtgattattcttgctacttcacagccttgccagacccctgatgactttgtggctgccacttctccgggacatatcataattggaggtttgtttgctattcatgaaaaaatgttgtcctcagaagactctcccagacgaccacaaatccaggagtgtgttggctttgaaatatcagtttttcttcaaactcttgccatgatacacagcattgagatgatcaacaattcaacactcttacctggagtcaaactggggtatgaaatctatgacacttgtacagaagtcacagtggcaatggcagccactctgaggtttctttctaaattcaactgctccagagaaactgtggagtttaagtgtgactattccagctacatgccaagagttaaggctgtcataggttctgggtactcagaaataactatggctgtctccaggatgttgaatttacagctcatgccacaggtgggttatgaatcaactgcagaaatcctgagtgacaaaattcgctttccttcatttttacggactgtgcccagtgacttccatcaaattaaagcaatggctcacctgattcagaaatctggttggaactggattggcatcataaccacagatgatgactatggacgattggctcttaacacttttataattcaggctgaagcaaataacgtgtgcatagccttcaaagaggttcttccagcctttctttcagataataccattgaagtcagaatcaatcggacactgaagaaaatcattttagaagcccaggttaatgtcattgtggtatttctgaggcaattccatgtttttgatctcttcaataaagccattgaaatgaatataaataagatgtggattgctagtgataattggtcaactgccaccaagattaccaccattcctaatgttaaaaagattggcaaagttgtagggtttgcctttagaagagggaatatatcctctttccattcctttcttcaaaatctgcacttgcttcccagtgacagtcacaaactcttacatgaatatgccatgcatttatctgcctgcgcatatgtcaaggacactgatttgagtcaatgcatattcaatcattctcaaaggactttggcctacaaggctaacaaggctatagaaaggaacttcgtcatgagaaatgacttcctctgggactatgctgagccaggactcattcatagtattcagcttgcagtgtttgcccttggttatgccattcgggatctgtgtcaagctcgtgactgtcagaaccccaacgcctttcaaccatgggagttacttggtgtgctaaaaaatgtgacattcactgatggatggaattcatttcattttgatgctcacggggatttaaatactggatatgatgttgtgctctggaaggagatcaatggacacatgactgtcactaagatggcagaatatgacctacagaatgatgtcttcatcatcccagatcaggaaacaaaaaatgagttcaggaatcttaagcaaattcaatctaaatgctccaaggaatgcagtcctgggcaaatgaagaaaactacaagaagtcaacacatctgttgctatgaatgtcagaactgtcctgaaaatcattacactaatcagacagatatgcctcactgccttttatgcaacaacaaaactcactgggcccctgttaggagcactatgtgctttgaaaaggaagtggaatatctcaactggaatgactccttggccatcctactcctgattctctccctactgggaatcatatttgttctggttgttggcataatatttacaagaaacctgaacacacctgttgtgaaatcatccgggggattaagagtctgctatgtgatccttctctgtcatttcctcaattttgccagcacgagctttttcattggagaaccacaagacttcacatgtaaaaccaggcagacaatgtttggagtgagctttactctttgcatctcctgcattttgacgaagtctctgaaaattttgctagccttcagctttgatcccaaattacagaaatttctgaagtgcctctatagaccgatccttattatcttcacttgcacgggcatccaggttgtcatttgcacactctggctaatctttgcagcacctactgtagaggtgaatgtctccttgcccagagtcatcatcctggagtgtgaggagggatccatacttgcatttggcaccatgctgggctacattgccatcctggccttcatttgcttcatatttgctttcaaaggcaaatatgagaattacaatgaagccaaattcattacatttggcatgctcatttacttcatagcttggatcacattcatccctatctatgctaccacatttggcaaatatgtaccagctgtggagattattgtcatattaatatctaactatggaatcctgtattgcacattcatccccaaatgctatgttattatttgtaagcaagagattaacacaaagtctgcctttctcaagatgatctacagttattcttcccatagtgtgagcagcattgccctgagtcctgcttcactggactccatgagcggcaatgtcacaatgaccaatcccagctctagtggcaagtctgcaacctggcagaaaagcaaagatcttcaggcacaagcatttgcacacatatgcagggaaaatgccacaagtgtatctaaaactttgcctcgaaaaagaatgtcaagtatatgaataagccttaggagatgccacattccagaataaaatgtttccagggtctttgcatct

SEQ ID NO: 29 - protein, 926 aa, Homo sapiens
MAFLIILITCFVIILATSQPCQTPDDFVAATSPGHIIIGGLFAIHEKMLSSEDSPRRPQIQECVGFEISVFLQTLAMIHSIEMINNSTLLPGVKLGYEIYDTCTEVTVAMAATLRFLSKFNCSRETVEFKCDYSSYMPRVKAVIGSGYSEITMAVSRMLNLQLMPQVGYESTAEILSDKIRFPSFLRTVPSDFHQIKAMAHLIQKSGWNWIGIITTDDDYGRLALNTFIIQAEANNVCIAFKEVLPAFLSDNTIEVRINRTLKKIILEAQVNVIVVFLRQFHVFDLFNKAIEMNINKMWIASDNWSTATKITTIPNVKKIGKVVGFAFRRGNISSFHSFLQNLHLLPSDSHKLLHEYAMHLSACAYVKDTDLSQCIFNHSQRTLAYKANKAIERNFVMRNDFLWDYAEPGLIHSIQLAVFALGYAIRDLCQARDCQNPNAFQPWELLGVLKNVTFTDGWNSFHFDAHGDLNTGYDVVLWKEINGHMTVTKMAEYDLQNDVFIIPDQETKNEFRNLKQIQSKCSKECSPGQMKKTTRSQHICCYECQNCPENHYTNQTDMPHCLLCNNKTHWAPVRSTMCFEKEVEYLNWNDSLAILLLILSLLGIIFVLVVGIIFTRNLNTPVVKSSGGLRVCYVILLCHFLNFASTSFFIGEPQDFTCKTRQTMFGVSFTLCISCILTKSLKILLAFSFDPKLQKFLKCLYRPILIIFTCTGIQVVICTLWLIFAAPTVEVNVSLPRVIILECEEGSILAFGTMLGYIAILAFICFIFAFKGKYENYNEAKFITFGMLIYFIAWITFIPIYATTFGKYVPAVEIIVILISNYGILYCTFIPKCYVIICKQEINTKSAFLKMIYSYSSHSVSSIALSPASLDSMSGNVTMTNPSSSGKSATWQKSKDLQAQAFAHICRENATSVSKTLPRKRMSSI

SEQ ID NO: 30 - DNA, 20 bp, Artificial Sequence
  Description of Artificial Sequence Synthetic primer
caggagtgtgttggctttga

SEQ ID NO: 31 - DNA, 20 bp, Artificial Sequence
  Description of Artificial Sequence Synthetic primer
atcaggtgagccattgcttt

SEQ ID NO: 32 - DNA, 26 bp, Artificial Sequence
  Description of Artificial Sequence Synthetic primer
cgggatccagacgaccacaaatccag

SEQ ID NO: 33 - DNA, 26 bp, Artificial Sequence
  Description of Artificial Sequence Synthetic primer
ccaagcttgattcataactcacctgt

SEQ ID NO: 34 - DNA, 21 bp, Artificial Sequence
  Description of Artificial Sequence Synthetic primer
cctggcttccgcaacttacac

SEQ ID NO: 35 - DNA, 25 bp, Artificial Sequence
  Description of Artificial Sequence Synthetic primer
gaccccttcattgacctcaactaca

SEQ ID NO: 36 - DNA, 24 bp, Artificial Sequence
  Description of Artificial Sequence Synthetic primer
ggtcttactccttggaggccatgt

(19) United States
(12) Patent Application Publication
(10) Pub. No.: US 2009/0142323 A1
(43) Pub. Date: Jun. 4, 2009

(54) METHODS FOR TREATING A DISORDER BY REGULATING GPRC6A

(76) Inventors: L. Darryl Quarles, Kansas City, KS (US); Min Pi, Overland Park, KS (US)

Correspondence Address: Workman Nydegger;1000 Eagle Gate Tower, 60 East South Temple, Salt Lake City, UT, 84111, US

(21) Appl. No.: 12/324,406

(22) Filed: Nov. 26, 2008

Related U.S. Application Data
(60) Provisional application No. 60/991,188, filed on Nov. 29, 2007.

(51) Int. Cl. A61K 31/56; A61K 38/43; A01K 67/027; C40B 30/06; A61P 35/00; A61P 3/00; C12Q 1/68; A61K 31/7105

(52) U.S. Cl. 424/94.1; 514/44; 514/169; 800/18; 435/6; 506/10

(57) ABSTRACT

A disorder related to a non-genomic androgen response or a metabolic syndrome can be treated, inhibited, and/or prevented by regulating an expression level and/or activity of GPRC6A. Such a method can include identifying an individual with a disorder associated with a non-genomic androgen response or metabolic syndrome; and administering to the individual in need thereof an agent capable of regulating an expression level and/or activity of GPRC6A thereby treating the disorder associated with the non-genomic androgen response or metabolic syndrome. The regulation of GPRC6A can increase or decrease the concentration of a sex hormone within said individual, as needed for a particular disease. Such regulating can also be used to treat, inhibit, or prevent the symptoms of such a disease.

### CROSS-REFERENCE TO RELATED APPLICATIONS

This patent application claims benefit of U.S. Patent Application Ser. No. 60/991,188, filed Nov. 29, 2007, which provisional application is incorporated herein by specific reference in its entirety.

The U.S. Government has a paid-up license in this invention and the right in limited circumstances to require the patent owner to license others on reasonable terms as provided for by the terms of grant NIH R01-AR37308 (L. D. Q.) awarded by the National Institute of Health, and COBRE grant P20 RR017686 (M. P.).

### BACKGROUND

The classical genomic actions of sex steroid hormones (e.g., estrogen, progesterone, and androgens) are carried out by steroid binding to specific nuclear receptors, translocation of the steroid-receptor complex to the nucleus, and binding of this complex to steroid response elements in promoters, thereby modulating gene expression over a period of hours. Sex steroid hormones are also known to have non-genomic effects. Non-genomic effects of sex steroids are mediated by steroid hormones binding to cell membranes leading to rapid cellular responses. These cellular responses can be mediated by several potential mechanisms, including translocation of steroid receptors to the cell surface membrane, nonspecific effects of steroids on the fluidity of lipids in the plasma membrane, direct allosteric modification of ligand-gated ion channels, and activation of G-protein coupled receptors (GPCRs).

GPRC6A is a recently identified member of family C of G protein-coupled receptors (GPCRs), and is thought to be most closely related to the calcium-sensing receptor (CASR). Structural homologies and conservation of specific domains in members of this family of receptors suggest an evolutionary link between extracellular calcium and amino acid-sensing. Indeed, GPRC6A has recently been shown to sense extracellular cations and amino acids, and may require both extracellular cations and amino acids for optimal stimulation in vitro. This dual sensitivity of GPRC6A to both divalent cations and amino acids is analogous to the related receptor CASR. However, compared to CASR, much higher extracellular calcium concentrations are needed to activate GPRC6A, and some studies suggest that cations may only be allosteric modulators of GPRC6A, whereas other studies show cation-dependent activation of GPRC6A. The calcimimetic NPS-R578, an allosteric modulator of CASR, and osteocalcin, a bone derived calcium binding protein, both enhance the functional responses of GPRC6A to extracellular calcium in vitro. The physiologically relevant ligands for and biological function of GPRC6A remain to be determined.

GPRC6A is broadly expressed in many tissues and organs, including lung, liver, spleen, heart, kidney, skeletal muscle, testis, brain, and bone. The amino acid, osteocalcin, and divalent calcium ligand interaction with this receptor and its wide tissue distribution implicate GPRC6A multiple processes. For example, GPRC6A may be a candidate for the elusive extracellular calcium-sensing mechanism known to be present in osteoblasts, which respond to high local Ca²⁺ concentrations (in the range of 8 to 40 mM), amino acids and osteocalcin in the bone microenvironment. GPRC6A is also a candidate for the putative osteocalcin receptor regulating energy metabolism.

Various methods for regulating GPRC6A expression and/or activity and detecting GPRC6A are described in Ekema, U.S. Published Patent Application No. 2004/0081970, which is incorporated by reference in its entirety. However, the function of GPRC6A and its physiological ligands have not been previously established.

### SUMMARY

In one embodiment, the present invention can include a method for treating, inhibiting, and/or preventing a disorder in an individual by regulating an expression level and/or activity of GPRC6A. Such a method can include identifying an individual with a disorder associated with a non-genomic androgen response or metabolic syndrome; and administering to the individual in need thereof an agent capable of regulating an expression level and/or activity of GPRC6A thereby treating the disorder associated with the non-genomic androgen response or metabolic syndrome. The regulation of GPRC6A can increase or decrease the concentration of a sex hormone within said individual, as needed for a particular disease. Such regulating can also be used to treat, inhibit, or prevent the symptoms of such a disease.

In one embodiment, the regulating is upregulating the expression level and/or activity of said GPRC6A. Such upregulating can be effected by administering to the individual an androgenergic agonist of GPRC6A.

In one embodiment, the disorder is an estrogen responsive breast cancer or ovarian cancer. In a treatment for estrogen responsive breast cancer or ovarian cancer, upregulation of GPRC6A can reduce the concentration of estradiol in the individual.

In one embodiment, the disorder is osteoporosis or osteopenia. The therapy can be provided by upregulating GPRC6A so as to increase bone density in the individual.

In one embodiment, the disorder is an metabolic syndrome. The therapy can be provided by upregulating GPRC6A so as to increase lean body mass and/or decreases body fat mass in the individual.

In one embodiment, the disorder is diabetes, which therapy is provided by upregulating GPRC6A.

In one embodiment, the upregulating can be effected by at least one approach selected from the group consisting of: (a) expressing in cells of said individual an exogenous polynucleotide encoding at least a functional portion of GPRC6A; (b) increasing expression of endogenous GPRC6A in said individual; (c) increasing endogenous GPRC6A activity in said individual; (d) introducing an exogenous polypeptide including at least a functional portion of GPRC6A to said individual; and (e) administering GPRC6A-expressing cells into said individual.

In one embodiment, the regulating is downregulating the expression level and/or activity of GPRC6A. Such downregulating can be effected by administering to the individual an androgenergic antagonist (i.e., anti-androgenergic) of GPRC6A. Examples of diseases that can be treated, inhibited, or prevented by downregulation of GPRC6A can include prostate cancer, benign prostatic hypertrophy, and the like.

In one embodiment, the downregulation of GPRC6A can be effected by administering to individual an agent selected from the group consisting of: (a) a molecule that binds said GPRC6A; (b) an enzyme which cleaves said GPRC6A; (c) an antisense polynucleotide capable of specifically hybridizing with at least part of an mRNA transcript encoding GPRC6A; (d) a ribozyme which specifically cleaves at least part of an mRNA transcript encoding GPRC6A; (e) a small interfering RNA (siRNA) molecule which specifically cleaves at least part of a transcript encoding GPRC6A; (f) a non-functional analogue of at least a catalytic or binding portion of said GPRC6A; and (g) a molecule which prevent GPRC6A activation or substrate binding.

In one embodiment, the present invention can include a method for upregulating GPRC6A in a subject. Such a method can includes administering to the subject an androgenergic agonist of said GPRC6A in a therapeutically effective amount to upregulate GPRC6A. For example, the androgenergic agonist can be selected from the group consisting of androgens, steroid hormones, androgenic hormones, anabolic steroids, testoids, testosterones, 19-carbon steroids, dehydroepiandrosterone (DHEA), dehydroepiandrosterone sulfate (DHEA-S), androstenedione, androstenediones, androstenediol, androsterone, dihydrotestosterone, androstanolone, fluoxymesterone, mesterolone, methyltestosterone, selective androgen receptor modulators (SARM), andarine, BMS-564,929, LGD-226, ostarine, S-40503, brimonidine tartrate, dexamethasone, indeloxazine hydrochloride, salts thereof, combinations thereof, and the like.

In one embodiment, the present invention can include a method for downregulating GPRC6A in a subject. Such a method can include administering to the subject an androgenergic antagonist of said GPRC6A in a therapeutically effective amount to downregulate GPRC6A. For example, the androgenergic antagonist can be selected from the group consisting of allylestrenol, oxendolone, osaterone acetate, bicalutamide, steroidal anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, combinations thereof, and the like.

In one embodiment, the present invention can include a GPRC6A knockout mouse having a GPRC6A gene having a deleted exon 2. The mouse can be heterozygous GPRC6A^{±} or homozygous GPRC6A^{-/-}.

In one embodiment, the present invention provides a method for identifying a substance that modulates GPRC6A. Such a method can include: providing a cell expressing GPRC6A; and screening the substance against the cell so as to determine whether or not the substance modulates GPRC6A. This can also include screening a library of substances. Substances that can be identified are those that upregulate or downregulate GPRC6A. The cell can naturally produce GPRC6A or can be transformed to a cell that expresses GPRC6A.

These and other embodiments and features of the present invention will become more fully apparent from the following description and appended claims, or may be learned by the practice of the invention as set forth hereinafter.

### FIGURES

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which: FIG. 1 includes

FIG. 1A is a schematic representation of a GPRC6A-deficient mouse model created by replacing exon 2 of the GPRC6A gene with the hygromycin resistance gene.

FIG. 1B is a picture of a PCR gel that shows the presence or absence of exon 2 in Wild-type GPRC6A^{+/+}, heterozygous GPRC6A^{±}, and homozygous GPRC6A^{-/-} mice.

FIG. 1C is a picture of an RT-PCR gel that shows GPRC6A expression in the kidney of GPRC6A^{+/+} but not in GPRC6A^{-/-} mice.

FIG. 1D is a picture of a Western Blot gel that shows GPRC6A expression in the kidney of GPRC6A^{+/+} but not in GPRC6A^{-/-}mice.

FIG. 2A is a picture showing the gross appearance of male GPRC6A^{-/-} mice, where the genito-anal distance is demarcated by arrows.

FIG. 2B is a graph illustrating a comparison of the genito-anal distance in 16 week-old GPRC6A^{+/+} and GPRC6A^{-/-} male mice.

FIG. 2C is a picture showing the gross appearance of testes of male GPRC6A^{+/+} and GPRC6A^{-/-} at age of 16 week-of-age, where the upper panel shows testis and epididymis (magnification 10×) and the lower panel shows dissected testis (magnification 20×) viewed under dissecting microscope.

FIG. 2D is a graph illustrating a comparison of testicular weight in 16 week-old GPRC6A^{+/+} and GPRC6A^{-/-} mice.

FIG. 2E is a graph illustrating a comparison of seminal vessicle weight in 16 week-old GPRC6A^{+/+} and GPRC6A^{-/-} mice.

FIG. 2F includes photographs of histological analysis of testes of 16 week-old GPRC6A^{+/+} and GPRC6A^{-/-} mice, which snow no abnormality, where the arrow heads depict sites of high GPRC6A expression in Leydig cells, and the arrows indicate that GPRC6A is also expressed in lower amounts in sertoli cells, spermatogonia and spermatids.

FIG. 2G is a photograph of mammary glands of 16 week-old GPRC6A^{+/+} and GPRC6A^{-/-} mice, which show abnormalities in GPRC6A^{-/-} mice.

FIG. 2H is a graph illustrating an increase in mammary fat pad mass in GPRC6A^{-/-} mice.

FIG. 2I is a graph illustrating serum testosterone of male and female GPRC6A^{+/+} and GPRC6A^{-/-} mice.

FIG. 2J is a graph illustrating serum estradiol of male and female GPRC6A^{+/+} and GPRC6A^{-/-} mice.

FIG. 2K is a graph illustrating serum FSH of male GPRC6A^{+/+} and GPRC6A^{-/-} mice.

FIG. 2L is a graph illustrating serum LH of male GPRC6A^{+/+} and GPRC6A^{-/-} mice.

FIG. 3A shows a RT-PCR analysis of androgen receptor (AR) expression, where AR expression in testis (Te) and bone marrow (BM) was not different between GPRC6A^{+/+} and GPRC6A^{-/-} male mice.

FIG. 3B is graph of a RT-PCR analysis of androgen receptor (AR) expression, where AR expression in testis (Te) and bone marrow (BM) was not different between GPRC6A^{+/+} and GPRC6A^{-/-} male mice.

FIG. 3C is a graph of a real time RT-PCR analysis of aromatase expression in testis.

FIG. 3D is a Western blot analysis of a comparison of the aromatase protein expression in testis from GPRC6A^{+/+} and GPRC6A^{-/-} male mice.

FIG. 3E is a photograph of immunohistochemistry showing in aromatase (CPY19) localized to Leydig cells (L, arrow head) and to a lesser degree in sertoli cells (SC), and spermatogonia (SG) (respectively indicated by arrows).

FIGS. 3F and 3G are graphs that show the real time RT-PCR analysis of Cyp17 and Sult1e1 expression in testis, respectively.

FIG. 3H is a RT-PCR analysis of GnRH expression in brain GPRC6A^{+/+} and GPRC6A^{-/-} male mice.

FIG. 3I is a graph of real time RT-PCR analysis of GnRH expression in brain GPRC6A^{+/+} and GPRC6A^{-/-} male mice.

FIG. 4A is a photograph that shows expression of GPRC6A messenger in kidney by in-situ hybridization, showing localization of both proximal and distal tubular segments.

FIG. 4B is a photograph of immunohistochemistry that shows NaPi IIa protein expression and translocation to the brush border membrane in GPRC6A^{-/-} mice.

FIG. 4C is a photograph of a RT-PCR gel analysis that shows the loss of GPRC6A resulted in decreased NaPi IIa message expression.

FIG. 4D is a graph of real time RT-PCR analysis that shows the loss of GPRC6A resulted in decreased NaPi IIa message expression (The arrow indicates β2-microglobulin).

FIG. 4E is a Western blot showing an increase in urinary excretion of a low molecular weight protein in GPRC6A^{-/-} mice identified as β2-microglobulin.

FIG. 4F is an immunoblot that identified the low molecular weight protein in GPRC6A^{-/-} mice as β2-microglobulin by immunobloting with an anti-β2-microglobulin antibody (The arrow indicates β2-microglobulin).

FIG. 5A includes photographs of a histological examination of the liver from GPRC6A^{+/+} and GPRC6A^{-/-} male mice at age of 16 week old by H & E stained (left panel), Oil Red O stained (right panel).

FIG. 5B is a graph illustrating hepatic triglyceride levels in GPRC6A^{+/+} and GPRC6A^{-/-} male mice at age of 16 week old.

FIG. 5C is a graph illustrating a glucose tolerance test (GTT) in 3 month-old male GPRC6A^{+/+} and GPRC6A^{-/-} mice.

FIG. 5D is a graph illustrating a insulin tolerance test (ITT) in 3 month-old male GPRC6A^{+/+} and GPRC6A^{-/-} mice.

FIG. 6A is a graph illustrating a comparison of the lean body mass of 6, 8, 12, and 16 week old male and female GPRC6A^{+/+} and GPRC6A^{-/-} mice.

FIG. 6B is a graph illustrating a comparison of the fat percent of 16 week old male and female GPRC6A^{+/+} and GPRC6A^{-/-} mice.

FIG. 6C is a graph illustrating a comparison of the femur bone mass density (BMD) of 6, 8, 12, and 16 week old male and female GPRC6A^{+/+} and GPRC6A^{-/-} mice.

FIG. 6D is an image of backscattered scanning electron microscopy analysis of tibia cortical bone in 16-week-old GPRC6A^{+/+} (upper panel) and GPRC6A^{-/-} mice (lower panel), where the arrows showed the diminished mineralization layer in the bone of GPRC6A^{-/-} mice.

FIG. 6E is an image of toluidine blue-stained plastic sections of femur from 16-week-old GPRC6A^{+/+} (upper panel) and GPRC6A^{-/-} mice (lower panel), where the arrows showed the unmineralized osteoid surfaces in the bone of GPRC6A^{-/-} mice.

FIG. 6F is an image of plastic unstained sections of tibia cortical bone viewed under fluorescent light in 16-week-old GPRC6A^{+/+} (upper panel) and GPRC6A^{-/-} mice (lower panel) prelabeled with twice calcein (double label).

FIG. 6G shows alkaline phosphatase (ALP) expression that was measured by RT-PCR from 4- and 10-day primary osteoblasts cultures derived from 8-week GPRC6A^{+/+} and GPRC6A^{-/-} mouse calvaria.

FIG. 6H shows alkaline phosphatase (ALP) activity in BMSCs from wild-type and GPRC6A^{-/-} mice cultured for 10 and 14 days.

FIG. 6I shows alizarin Red-S for GPRC6A^{+/+} and GPRC6A^{-/-} showing mineralization of extracellular matrix.

FIG. 7A shows the GPRC6A response to extracellular steroid hormones, testosterone and synthetic androgen (R1881), which stimulated the GPRC6A-mediated activation of phospho-ERK (upper panel); as control the HEK293 (middle panel) and HEK293 transfected calcium sensing receptor (CASR) cells (lower panel) did not responded to the testosterone and R1881.

FIG. 7B shows that testosterone-BSA stimulated the GPRC6A mediated activation of phospho-ERK.

FIG. 7C shows that the non-steroidal anti-androgen, flutamide did not inhibited testosterone stimulated the GPRC6A mediated activation of phospho-ERK.

FIG. 7D shows that testosterone stimulated the GPRC6A mediated activation of phospho-ERK in both the cytosol and nucleus.

FIG. 7E shows that extracellular calcium is required for GPRC6A sensing of testosterone.

FIG. 7F shows that dehydroandrosterone (DHEA), beta-estradiol, cholesterol, 1,25(OH)2VitD3, and dexamethasone, but not progesterone stimulated GPRC6A-mediated activation of phospho-ERK.

FIG. 7G shows that the surface binding of testosterone-BSA-FITC was present in HEK293 cells transfected with GPRC6A, but not in untransfected HEK293 cells (the nuclei were stained by DAPi).

FIG. 7H includes a picture of a RT-PCR gel that indicates GPRC6A and AR did not expressed in HEK-293 cells by RT-PCR.

FIG. 7I includes a graph that shows synthetic androgen (R1881) stimulated the GPRC6A mediated activation of luciferase when HEK-293 cells were co-transfected with pcDNA3.mGPRC6A and SRE-luciferase reporter gene plasmid.

FIG. 7J includes a graph that shows testosterone binding to a membrane fraction of HEK-293 cells transfected with GPRC6A.

FIGS. 8A-8C show the response to testosterone in GPRC6A knockout mice, where BMSCs derived from the male GPRC6A^{/}− mice exhibited a reduced ability to activate ERK in response to testosterone (80 nM), extracellular calcium, and the calcimimetics, NPS-R568, respectively, as assessed by Western blot analysis using an antiphospho-ERK antibody.

FIGS. 8D-8E show the impact of the loss of GPRC6A on the capacity of testosterone to stimulate phospho-ERK activity and early growth-responsive 1 (Egr-1) expression in bone marrow and testes in vivo.

FIG. 9A is a picture of a gel that indicates R1881 stimulated GPRC6A mediated non-genomic activation of intercellular phospho-Src and phospho-Raf-1.

FIG. 9B is a picture of a gel that shows testosterone and β-estradiol stimulated GPRC6A-mediated activation of phospho-ERK were each blocked by 100 ng/ml Pertussis toxin (PTx).

FIG. 9C is a picture of a gel that shows testosterone stimulated GPRC6A-mediated activation of phospho-ERK which was inhibited by 10 μM PD89059, 50 μM Ly294002, 2 μM U73122 and 10 μM PP-1.

FIGS. 9D-9F include graphs that illustrate R1881, synthetic androgen stimulated the GPRC6A-mediated activation of luciferase were inhibited by PD89059 (FIG. 9D), Ro31-8220 (FIG. 9E) and PP-1 (FIG. 9F).

FIG. 9G is a schematic diagram of the signal transduction pathway of GPRC6A.

FIG. 10A is a picture of a gel that indicates BMSCs derived from the male GPRC6A^{-/-} mice exhibited a reduced ability to activate ERK in response to testosterone (80 nM).

FIG. 10B is an image of dissected seminal vesicle of wild-type and GPRC6A null mice after sham and castration (ORX) with or without testosterone replacement.

### DETAILED DESCRIPTION

The present invention relates to compositions and methods for treating, inhibiting, and/or preventing a disorder in a subject by regulating or modulating the G-protein coupled receptor GPRC6A or functionality thereof. The modulation of GPRC6A can achieve therapeutic states: (1) androgen or similar agonist can increase GPRC6A functionality to provide a non-genomic androgen response; (2) a GPRC6A antagonist can inhibit a non-genomic androgen response; (3) a GPRC6A agonist increase the activity of GPRC6A to increase anabolic responses in multiple tissues (e.g., bone, fat, muscle, liver, pancreas, kidney, and the like) with regard to a metabolic disorder; and (4) a GPRC6A antagonist can decrease the activity of GPRC6A to decrease an anabolic response in the tissues. As such, treating, inhibiting, and/or preventing the disorder can be carried out by regulating or modulating the amount or activity of GPRC6A in the subject. Accordingly, regulating or modulating can be performed by administering the subject a therapeutically effective amount of an androgenergic agonist, an androgenergic antagonist, or allosteric modulator such that the amount or activity of GPRC6A is regulated or modulated in accordance with the needed therapy for a particular disease state or symptoms thereof. Also, the therapy can be performed by increasing or decreasing the number of cell surface GPRC6A receptors that are available for binding an agonist, antagonist, or allosteric modulator.

### I. Introduction

GPRC6A is a widely expressed orphan G-protein coupled receptor that can sense extracellular amino acids, osteocalcin, and divalent cations. The entire scope of physiological functions of GPRC6A is unknown. In order to study GPRC6A, knockout mice were created and characterized to have the phenotype of GPRC6A^{-/-} mice. A complex multiorgan, metabolic-like syndrome was in GPRC6A^{-/-} mice that suggests that GPRC6A is involved in nutritional pathways coordinating the metabolic activity of multiple tissues in response to changes in extracellular amino acids and divalent cations. Complex metabolic abnormalities were found in GPRC6A^{-/-} mice involving multiple organ systems that express GPRC6A, including bone, kidney, testes, and liver were studied. GPRC6A^{-/-} mice exhibited hepatic steatosis, hyperglycemia, glucose intolerance, and insulin resistance. In addition, high expression levels of GPRC6A in Leydig cells in the testis were observed. GPRC6A was also highly expressed in kidney proximal and distal tubules, and GPRC6A^{-/-} mice exhibited increments in urine Ca/Cr and PO₄/Cr ratios as well as low molecular weight proteinuria. Finally, GPRC6A^{-/-} mice exhibited a decrease in bone mineral density (BMD) in association with impaired mineralization of bone.

GPRC6A^{-/-} mice have a metabolic syndrome characterized by defective osteoblast-mediated bone mineralization, abnormal renal handling of calcium and phosphorus, fatty liver, glucose intolerance, and disordered steroidogenesis. These findings suggest the overall function of GPRC6A may be to coordinate the anabolic responses of multiple tissues through the sensing of extracellular amino acids, osteocalcin and divalent cations.

It has now been found that GPRC6A, previously described as an amino acid and an extracellular calcium sensing receptor, mediates the non-genomic actions of androgens. In cells that overexpress GPRC6A, but lack the nuclear androgen receptor, GPRC6A localizes to the cell surface membrane, where it mediates testosterone binding and androgen-stimulated ERK activation. Ablation of GPRC6A in mice results in feminization, loss of lean body mass, osteopenia, and increased fat in association with increased circulating levels of estradiol, and reduced testosterone levels in males. In addition, GPRC6A^{-/-} mice display attenuation of testosterone-stimulated ERK activation and Egr-1 expression in bone marrow stromal cells in vitro and in target tissues in vivo. Taken together, these data provide the first evidence that the orphan receptor GPRC6A is a biologically relevant androgen sensor, and thereby related to non-genomic androgen response and metabolic syndromes.

Also, GPRC6A^{-/-} mice have a metabolic syndrome characterized by defective osteoblast-mediated bone mineralization, abnormal renal handling of calcium and phosphorus, fatty liver, glucose intolerance and disordered steroidogenesis, a phenotype resembling metabolic syndrome and Type II diabetes mellitus. These findings suggest the overall function of GPRC6A may be to coordinate the anabolic responses of multiple tissues through the sensing of extracellular amino acids, osteocalcin and divalent cations. Either pharmaceutical, genetic or biological approaches to activate or increase GPRC6A can be used as treatments for multiple organ dysfunction in metabolic syndrome.

### II. Therapeutic Methods

In one embodiment, the methods of the present invention can be used to treat, inhibit, and/or prevent disorders by regulating or modulating the expression, amount, or activity of GPRC6A to achieve a desired non-genomic androgen response or treat a metabolic syndrome in an individual. Such a method can include: (i) identifying an individual with or susceptible to a disorder associated with a non-genomic androgen response or metabolic disorder; and (ii) providing to the individual an agent capable of regulating or modulating an expression level, amount, and/or activity of GPRC6A in a therapeutically effective amount. In some instances in some disease states, the regulating or modulating can be a decrease. In other instances in some disease states, the regulating or modulating can be an increase.

In one embodiment, the method can include regulating or modulating the amount or activity of GPRC6A so as to increase or a decrease the concentration of a sex hormone within the individual. The regulating or modulating of the amount of GPRC6A can be carried out by upregulating or downregulating the expression level of GPRC6A by agents that target the promoter for GPRC6A. Biological agents that regulate the expression of GPRC6A can be identified or developed as described herein, and such agents can be used to modulate GPRC6A.

In one embodiment, upregulation of the amount or activity of GPRC6A can be effected by using one or more of the following techniques: (a) expressing in cells of said individual an exogenous polynucleotide encoding at least a functional portion of GPRC6A; (b) increasing expression of endogenous GPRC6A in said individual; (c) increasing endogenous GPRC6A activity in said individual; (d) introducing an exogenous polypeptide including at least a functional portion of GPRC6A to said individual; (e) administering GPRC6A-expressing cells into said individual; or (f) introducing the extracellular domain of GPRC6A to a cell so as to act as a dominant negative to disrupt function of the GPRC6A receptor.

For example, the upregulated expression level of GPRC6A can be effected by administration of a nucleic acid that encodes for GPRC6A. Examples of such a nucleic acid can include DNA that encodes GPRC6A, such as a plasmid like pcDNA3.mGPRC6A, a cDNA, or other encoding DNA. RNA that encodes GPRC6A can also be administered. GenBank provides the following accession numbers, which sequences are incorporated herein by specific reference: GPRC6A gene is NC_{—}000006 (SEQ ID NO: 28); and the protein sequence of human GPRC6A (hGPRC6A) is N 148963 (SEQ ID NO: 29).

In one embodiment, downregulation of the amount or activity GPRC6A can be effected by introducing into an individual one or more of the following agents: (a) a molecule that binds the GPRC6A; (b) an enzyme which cleaves the GPRC6A; (c) an antisense polynucleotide capable of specifically hybridizing with at least part of an mRNA transcript encoding GPRC6A; (d) a ribozyme which specifically cleaves at least part of an mRNA transcript encoding GPRC6A; (e) a small interfering RNA (siRNA) molecule which specifically cleaves at least part of a transcript encoding GPRC6A; (f) a non-functional analogue of at least a catalytic or binding portion of the GPRC6A; or (g) a molecule that prevent GPRC6A activation or substrate binding.

The regulating or modulating of the activity of GPRC6A can be carried out by increasing or decreasing the activity level of GPRC6A.

For example, upregulating the activity can be effected by administering to the individual an androgenergic agonist of the GPRC6A. Examples of androgenergic agonists include, androgens, steroid hormones, androgenic hormones, anabolic steroids, testoids, testosterones, 19-carbon steroids, dehydroepiandrosterone (DHEA), dehydroepiandrosterone sulfate (DHEA-S), androstenedione, androstenediones, androstenediol, androsterone, dihydrotestosterone, androstanolone, fluoxymesterone, mesterolone, methyltestosterone, selective androgen receptor modulators (SARM), andarine, BMS-564,929, LGD-226, ostarine, S-40503, brimonidine tartrate, dexamethasone, indeloxazine hydrochloride, salts thereof, combinations thereof, and the like.

In another example, downregulating the activity can be effected by administering to the an androgenergic antagonist or anti-androgenergic agent. Downregulating GPRC6A can be used to block androgen responses. Examples of downregulators of GPRC6A (e.g., androgenergic antagonist or anti-androgenergic agents) can include allylestrenol, oxendolone, osaterone acetate, bicalutamide, steroidal anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, combinations thereof, and the like.

GPRC6A is also activated by extracellular calcium, which has direct actions on multiple organs, including osteoblasts in bone, calcimimetics, amino acids, and osteocalcin the latter of which has recently been shown to be a bone derived factor that regulates energy metabolism. As such, calcium, calcimimetics, amino acids, and osteocalcin can be used to upregulate GPRC6A, and may function as allosteric modulators of GPRC6A.

Accordingly, the present invention can also include a pharmaceutical composition having a GPRC6A upregulating or downregulating agent. Such a composition can include a pharmaceutically acceptable carrier, such as those well known in the art, and a therapeutically effective amount of the agent.

### III. Diseases

GPRC6A is involved, through hormonal regulatory pathways, with metabolism of energy, fat, bone, and glucose. As such, upregulation of the amount or activity of GPRC6A can be used for treating, inhibiting, and/or preventing defective mineralization of bone, impaired osteoblast function, decreases in lean body mass, increases in fat mass, hyperphosphatemia, hypercalciuria, hyperglycemia, and feminization of males associated with altered ratio of estradiol and testosterone. Additionally, GPRC6A can be used for treating, inhibiting, and/or preventing elevated serum glucose levels, glucose intolerance, insulin resistance, and hepatic steatosis. A therapy for such disorders can include administering a therapeutically effective amount of an agent to increase GPRC6A amount or activity. As such, the amount or activity of GPRC6A can be increased by a therapeutically effective amount.

In one embodiment, the disorder to be treated, inhibited, and/or prevented can be osteoporosis or osteopenia. As such, upregulating the amount or activity of GPRC6A can be used to stimulate anabolic bone mass densification. Such bone mass densification can be used as a therapy for osteoporosis or osteopenia.

In one embodiment, the disorder to be treated, inhibited, and/or prevented can be an estrogen responsive breast cancer or ovarian cancer. The amount or activity of GPRC6A can be upregulated in a therapeutically effective amount to cause a reduction in the production of an estrogen, such as estradiol. Reducing estradiol has been shown to reduce cancer in breast cancer and ovarian cancer patients (e.g., using aromatase inhibitors). Thus, by upregulating the amount or activity of GPRC6A, estradiol concentrations can be lowered, thereby treating, inhibiting, and/or preventing estrogen responsive breast cancer and/or ovarian cancer.

In one embodiment, the disorder to be treated, inhibited, and/or prevented can be prostate cancer. Downregulating GPRC6A can be used to block androgen responses, which can be used to treat prostate cancer by reducing the levels of testosterone in an individual or in certain tissues of an individual.

In one embodiment, the disorder that can be treated, inhibited, and/or prevented by upregulating GPRC6A can be any metabolic syndrome, which benefits from an upregulation in lean body mass and/or down-regulating body fat mass. Examples of metabolic syndromes include obesity-dependent metabolic syndrome, insulin resistance syndrome, and the like.

In one embodiment, the disorder to be treated, inhibited, and/or prevented can be diabetes, which benefits from an upregulation in lean body mass and/or down-regulating body fat mass.

In one embodiment, the disorder to be treated, inhibited, and/or prevented can be benign prostatic hypertrophy. Such a therapy can be achieved by downregulating the amount or activity of GPRC6A either by pharmacological means or by a dominant negative biological agent derived from GPRC6A extracellular domain

In one embodiment, GPRC6A can be upregulated so as to induce feminization of male. Such feminization may be useful in certain circumstances, such as transgendered men.

In one embodiment, GPRC6A can be upregulated so as to increase lean body mass. Individuals that are underweight or that have eating disorders may obtain increased health benefits from an increase in lean body mass.

In one embodiment, GPRC6A can be downregulated so as to decrease lean body mass. Individuals that are overweight or that become slimmer may obtain increased health benefits from a decrease in lean body mass.

In one embodiment, GPRC6A can be upregulated so as to decrease body fat mass. Individuals that are overweight or that become slimmer may obtain increased health benefits from a decrease in body fat mass.

In one embodiment, GPRC6A can be downregulated so as to increase body fat mass. Individuals that are underweight or that have eating disorders may obtain increased health benefits from an increase in body fat mass.

### IV. Drug Screening.

In one embodiment, the present invention can include a method for identifying a substance that modulates GPRC6A. As such, a cell can be provided that expresses GPRC6A. A substance can then be screened against the cell so as to determine whether or not the substance modulates GPRC6A. The substance can be in a library of substances, and the entire library or portion thereof can be screened. Substances can be identified that upregulate GPRC6A or downregulate GPRC6A. Substances that are identified can be used in the therapies described herein. The cell can naturally express GPRC6A. Alternatively, the cell can be a cell that is transformed from a non-GPRC6A cell to a cell that expresses GPRC6A.

### Experimental

1.

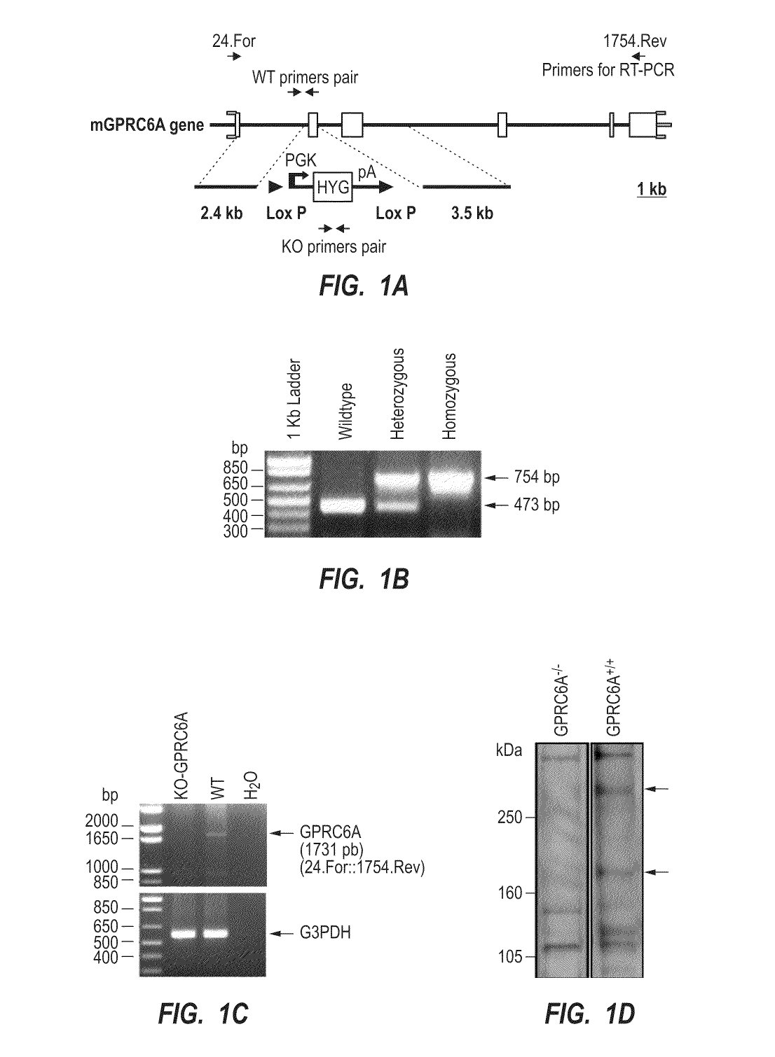

To address the function of GPRC6A in vivo, we selectively deleted exon 2 of the mouse GPRC6A gene. The GPRC6A-deficient mouse model was created by replacing exon 2 of the GPRC6A gene with the hygromycin resistance gene (FIG. 1A). To generate the targeting construct, the hygromycin resistance gene under the control of the PGK promoter was cloned into the Sma I and Eco RV sites of pBS-lox, which was produced by cloning the oligonucleotide Lox71: 5′-ctagataccgttcgtatagcatacattatacgaagttatg-3′ (SEQ ID NO: 1) into the Xba I and Bam HI sites and the oligonucleotide Lox 66: 5′-agcttataacttcgtatagcatacattatacgaacggtag-3′ (SEQ ID NO: 2) into the Hind III and Sal I sites of pBluescript (Stratagene), to produce pBS-lox-PGK-Hyg. A genomic fragment from intron 1 of GPRC6A gene, representing the 5′ homologous targeting region, was amplified by PCR using Advantage 2 Taq polymerase (BD Biosciences) and primers PP 232-Avr: 5′-aaacctagggccattcatgaaaaaatgttgtcctcagatgaccatcc-3′ (SEQ ID NO: 3), and PP 233-Avr: 5′-aaacctaggctcactcaacccccatgtccttccaactctagctg-3′ (SEQ ID NO: 4), digested with Avr II, and cloned into the Xba I site of pBS-lox-PGK-Hyg to produce pBS-GPRC6A-Intron 1. A genomic fragment from intron 2 of GPRC6A, representing the 3′ homologous targeting region, was then amplified by PCR using Advantage 2 Taq polymerase and primers 298: 5′-aaagtcgacctacattggtccatcgattacattagttcttgg-3′ (SEQ ID NO: 5) and PP 299: 5′-aaagtcgacgaggccttgaggtcaaactccagaaccccagag-3′ (SEQ ID NO: 6), digested with Sal I, and cloned into the Sal I site of pBS-GPRC6A-Intron I to produce pGPRC6A-KO. The methods for knocking out the GPRC6A gene have been described previously in detail (Svard J, Heby-Henricson K, Persson-Lek M, Rozell B, Lauth M, et al. (2006), Genetic elimination of Suppressor of fused reveals an essential repressor function in the mammalian Hedgehog signaling pathway; Dev Cell 10: 187-197).

Briefly, the mouse embryonic cell line RW-4, derived from 129X1/SvJ mouse strain (Hug B A, Wesselschmidt R L, Fiering S, Bender M A, Epner E, et al. (1996) Analysis of mice containing a targeted deletion of beta-globin locus control region 5′ hypersensitive site 3. Mol Cell Biol 16: 2906-2912), and kindly provided by Stephan Teglund at the Karolinska Institute Center for Transgene Technologies, was transfected by electroporation with Not I linearized pGPRC6A-KO. Hygromycin resistant embryonic stem cell colonies were picked, expanded, and tested by PCR to identify clones in which the PGK-Hygromycin gene had correctly replaced exon 2 of the GPRC6A gene. The correctly mutated embryonic stem clones were injected into blastocysts derived from C57BL/6 3.5 days after mating and implanted into B6CBAF1 pseudopregnant females. The resulting male chimeras were bred with female C57BL/6 mice. Homozygous founders were generated by mating the resulting heterozygous mice. The successful targeting of GPRC6A in embryonic stem (ES) cells was confirmed by Southern blot analysis of the genomic DNA from ES cell clones. We observed no apparent differences in the founders generated from different ES cell clones. We focused our studies on founder line 17.

We selectively deleted exon 2 of the mouse GPRC6A gene (FIG. 1A). Wild-type GPRC6A^{+/+}, heterozygous GPRC6A^{±}, and homozygous GPRC6A^{-/-} mice were genotyped by PCR (FIG. 1B) and each genotype was found to be born at the expected Mendelian frequencies. In addition, full-length GPRC6A transcripts and proteins were documented to be absent from various tissues of GPRC6A^{-/-} mice by RT-PCR (FIG. 1C) and Western Blot (FIG. 1D). GPRC6A^{-/-} mice (as well as heterozygous GPRC6A^{±} mice) were similar in gross appearance, body weight and body length to wild-type littermates (data not shown). There were no identified abnormalities in gait or physical activity between wild-type and GPRC6A^{-/-} mice. X-ray analysis indicated no gross abnormalities in the development of the skeleton in the GPRC6A^{-/-} mice (data not shown).

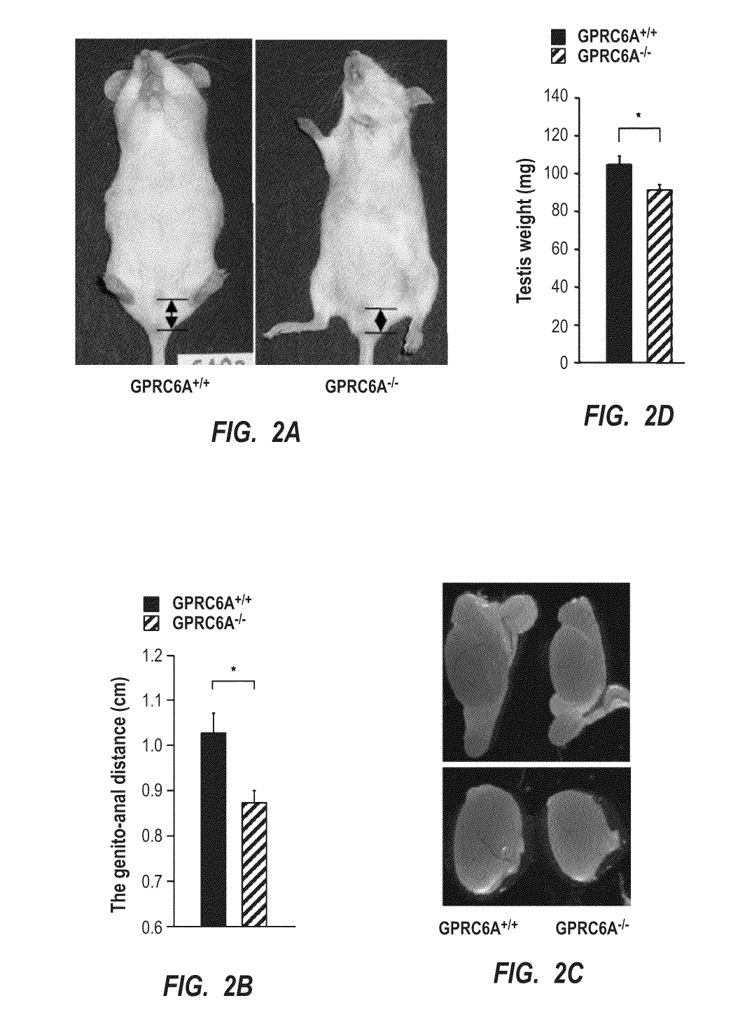

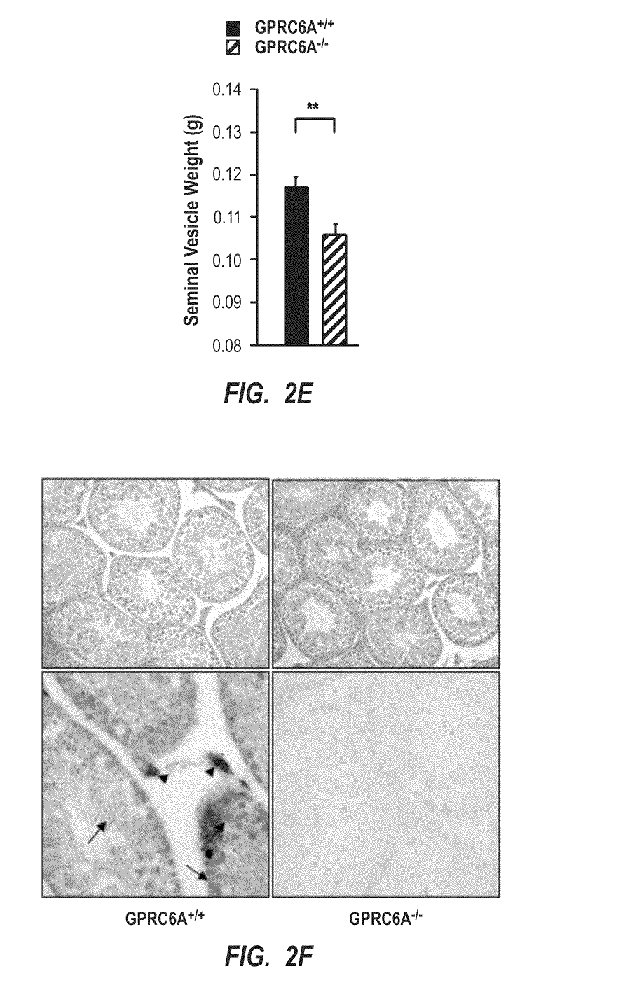

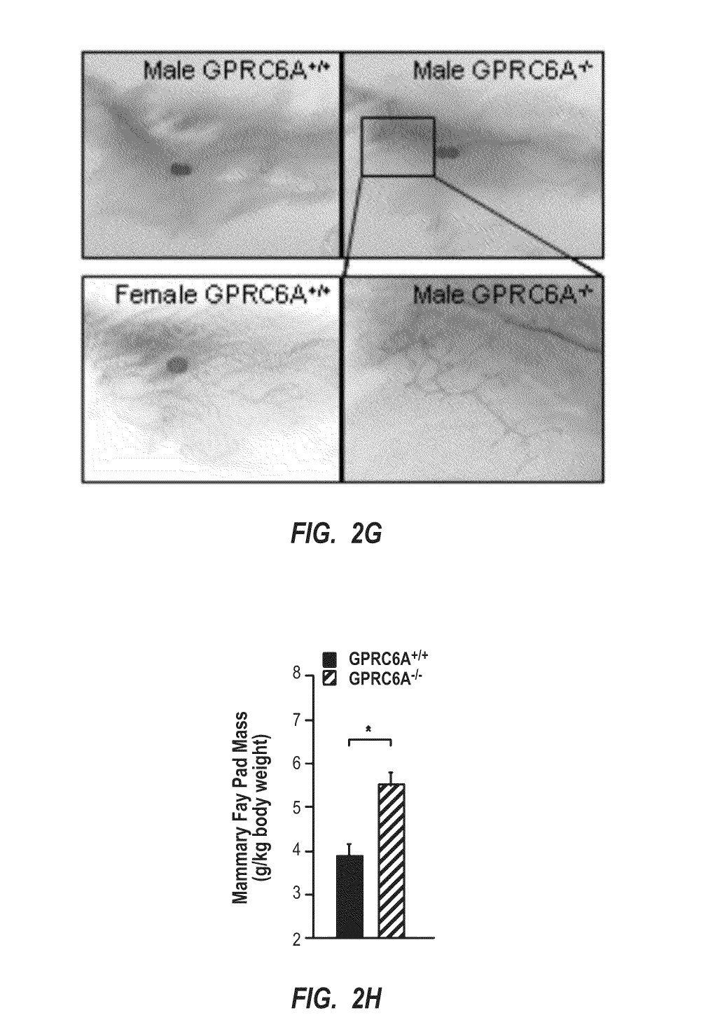

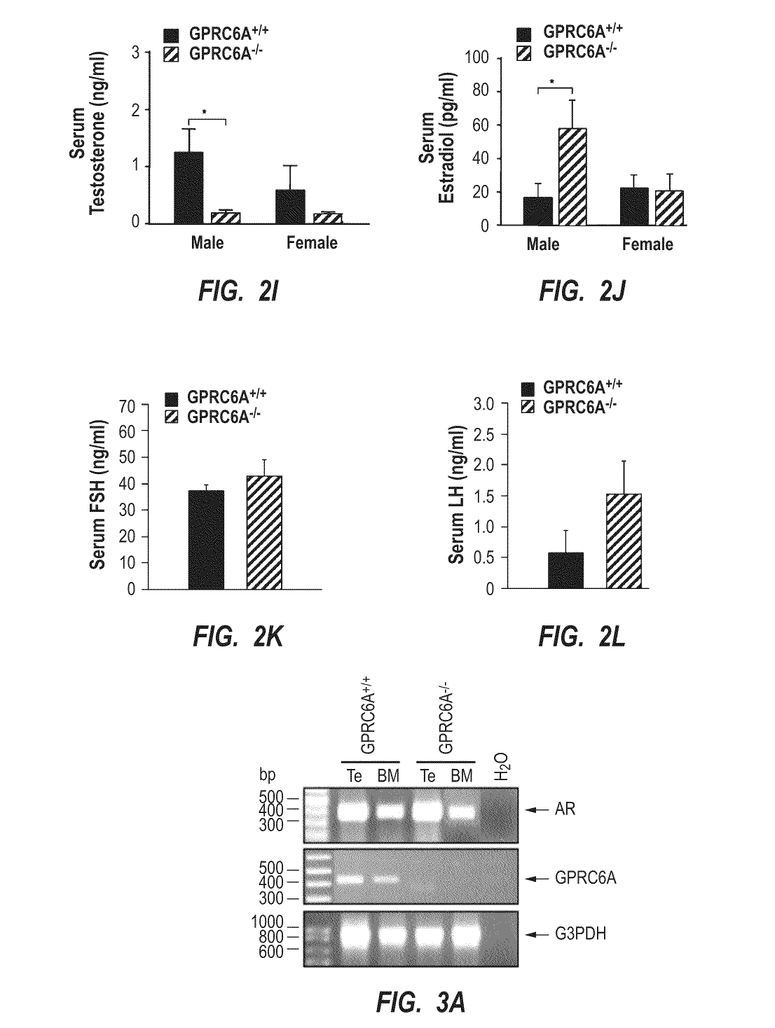

On closer inspection, we noted that male GPRC6A^{-/-} mice had feminization of the external genitals (FIGS. 2A-2C). In 16-week-old male mice, the genito-anal distance (FIGS. 2A and 2B) as well as testicular size (FIG. 2C), testicular weight (FIG. 2D) and the weight of seminal vesicle (FIG. 2E) were significantly reduced in GPRC6A^{-/-} compared to wild-type littermates. No histological abnormality of the testes was noted in GPRC6A^{-/-} mice (FIG. 2F). GPRC6A was highly expressed in Leydig cells, and was also expressed in sertoli cells, spermatogonia and spermatids by in-situ hybridization analysis.

2.

Mice mammary fat pads were excised and fixed for a minimum of 2 h in Carnoy's solution (60% ethanol, 30% chloroform, and 10% glacial acetic acid). The fixed glands were washed in 70% ethanol for 15 min and then rinsed in water for 5 min. The mammary glands were stained overnight at 4° C. in carmine alum stain (1 g carmine and 2.5 g aluminum potassium sulfate in 500 ml water).

We also found abnormalities of mammary glands in male GPRC6A^{-/-} mice, as evidence by greater ductal outgrowth in the mammary fat pad (in 10/14 GPRC6A^{-/-} compared to 3/13 mice wild-type male mice (FIG. 2G), and increased the mammary fat pad mass (FIG. 2H). We found no evidence of embryonic lethality or reduced fertility in homozygous null male or female mice when breed to their respective wild-type mates; however we observed a reduced litter size from breeding pairs consisting of both male and female homozygous null mice.

3.

We compared the serum testosterone, estradiol, follicle-stimulating hormone (FSH), and luteinizing hormone (LH) concentration in 16-week-old mice wild-type and GPR6CA^{-/-} mice. Testosterone concentrations in male GPRC6A knockout mice were significantly lower (FIG. 21) and the estradiol concentrations were significantly higher in male GPRC6A^{-/-} mice compared to wild-type littermates (FIG. 2J). Estradiol levels were not different between wild-type and GPRC6A^{-/-} female mice (FIG. 2J), although the circulating testosterone levels were lower in female GPRC6A null mice (FIG. 2I). Furthermore, serum follicle-stimulating hormone (FSH) and luteinizing hormone (LH) levels in male mice were not significantly different between wild-type and the GPRC6A^{-/-} male mice, FIG. 2K and FIG. 2L, respectively.

4.

Since inactivation of the androgen receptor is reported to lower testosterone levels in mice, we examined if loss of GPRC6A lowered androgen receptor expression. RT-PCR was performed using two-step RNA PCR (Perkin-Elmer) as previously described (Pi M, Faber P, Ekema G, Jackson P D, Ting A, et al. (2005) Identification of a novel extracellular cation-sensing G-protein-coupled receptor. J Biol Chem 280: 40201-40209). Specific intron-spanning primer sets were to amply the specified transcripts. For quantitative real-time RT-PCR assessment of bone markers expression, we isolated and reverse transcribed 2.0 μg total RNA from long bone of 8-week-old mice as previously described (Xiao Z S, Simpson L G, Quarles L D (2003) IRES-dependent translational control of Cbfa1/Runx2 expression. J Cell Biochem 88: 493-505). For quantitative real time RT-PCR assessment of aromatase, CYP17 and Sutllel genes expression we isolated and reverse transcribled total RNA isolated from testis, brain, fat, liver and pituitary of 33 week-old GPRC6A^{+/+} (n=5) and GPRC6A^{-/-} mice (n=5) as previously described (Hiroi H, Christenson L K, Chang L, Sammel M D, Berger S L, et al. (2004) Temporal and spatial changes in transcription factor binding and histone modifications at the steroidogenic acute regulatory protein (stAR) locus associated with stAR transcription. Mol Endocrinol 18: 791-806) using specific primer sets.

The following intron-spanning primer sets were used for RT-PCR: mGPRC6A.24. For: ccagaaagatggccctattga (SEQ ID NO: 7); mGPRC6A.1754.Rev: ctccttactggggcccagtggg (SEQ ID NO: 8); mAndR.F578: caacttgcatgtggatgacc (SEQ ID NO: 9) and mAndR.R961: cttgagcaggatgtgggattc (SEQ ID NO: 10). mGnRH.For169: agcactggtcctatgggttg (SEQ ID NO: 11) and mGnRH.Rev389: gggccagtgcatctacatct (SEQ ID NO: 12). NaPiII.F248: ccacctatgccatctccagt (SEQ ID NO: 13) and NaPiII.R635: accatgctgacaatgatgga (SEQ ID NO: 14); mALP.905F: aacccagacacaagcattcc (SEQ ID NO: 15) and mALP.1458R: ctgggcctggtagttgttgt (SEQ ID NO: 16), G3PDH.F143: gaccccttcattgacctcaactaca (SEQ ID NO: 17); G3PDH.R1050: ggtcttactccttggaggccatgt (SEQ ID NO: 18) for control RNA loading. The following primer sets were used for real-time PCR: aromatase forward primer: tgagaacggcatcatatttaacaac (SEQ ID NO: 19) and reverse primer: gcccgtcagagctttcataaag (SEQ ID NO: 20); Cyp17 forward primer: tggaggccactatccgagaa (SEQ ID NO: 21) and reverse primer: tgttagccttgtgtgggatgag (SEQ ID NO: 22); and Sultlel forward primer: tcatgcgaaagggaattatagga (SEQ ID NO: 23) and reverse primer: tgcttgtagtgctcatcaaatctct (SEQ ID NO: 24).

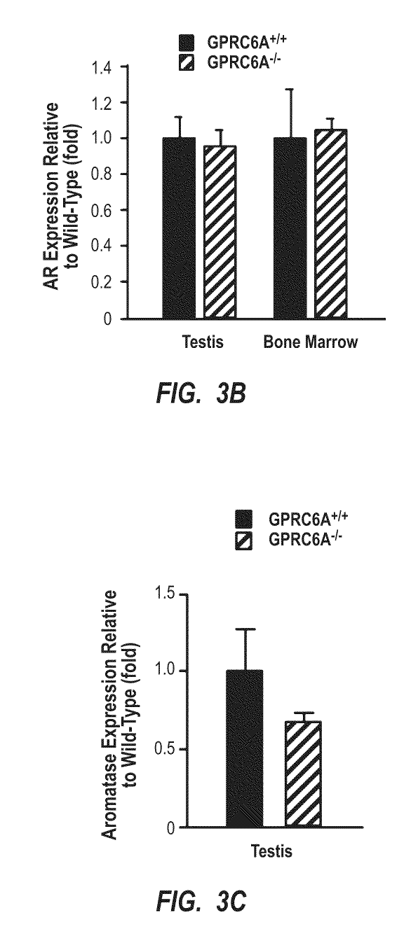

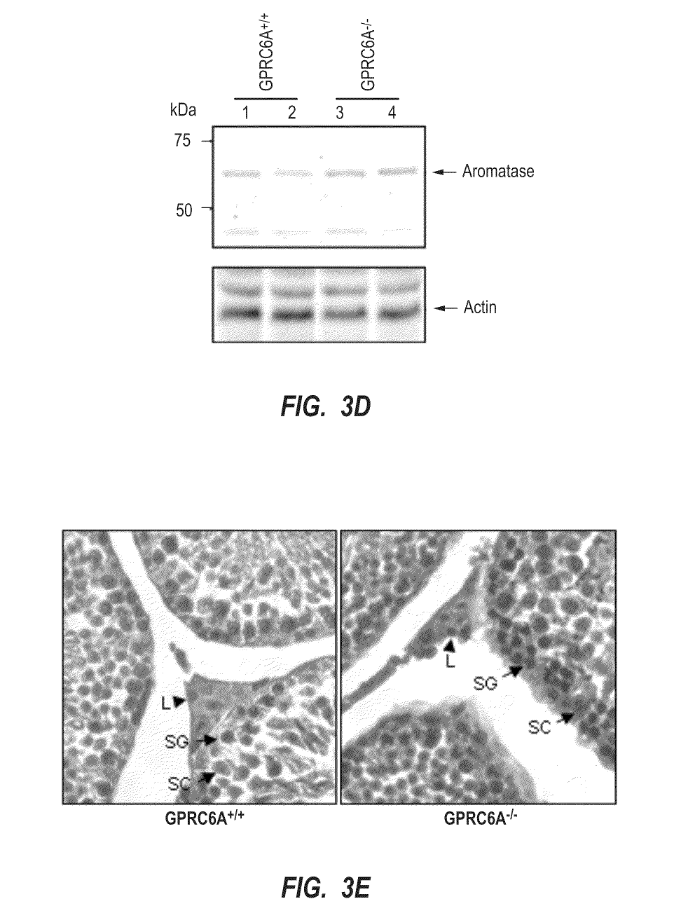

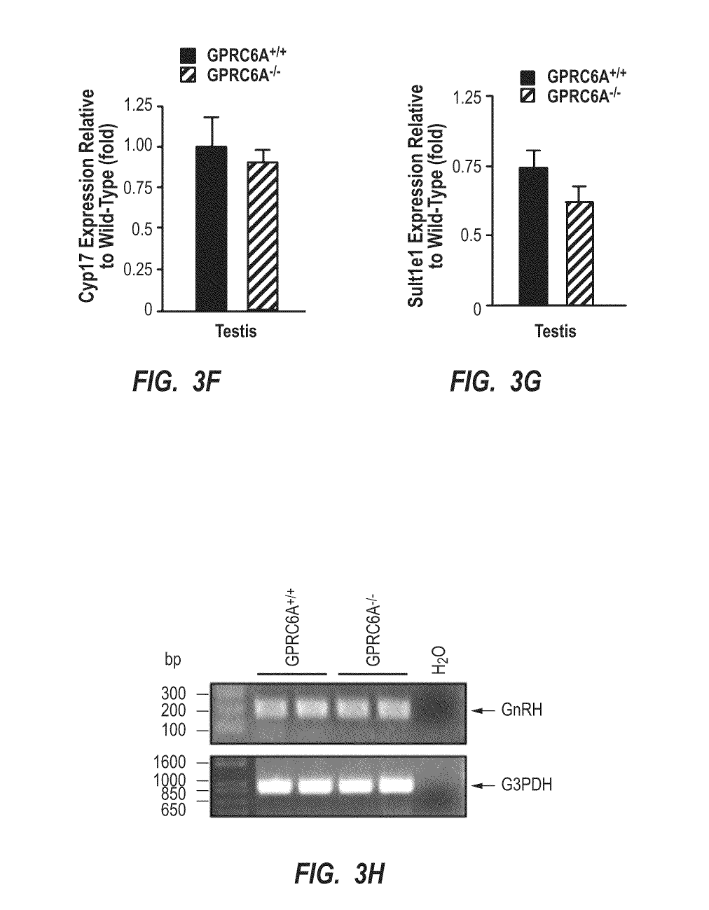

FIG. 3A shows that GPRC6A does not effect expression of the androgen receptor, providing further support for a direct role of GPRC6A in mediating androgen responses.

We observed no difference in AR transcripts in the testis and bone marrow by RT-PCR (FIG. 3B). To explore the possibility that the reduction in testosterone was due to increased aromatase-mediated conversion for testosterone to estrogen, we examined aromatase gene (Cyp19a1) expression by real-time RT-PCR. The quantitative real-time PCR using primers located in exon 4 and the interface between exons 4 and 5 failed to document any difference in the expression of Cyp19a1 in GPRC6A null mice testis (FIG. 3C).

We did, however, detect a small increase in aromatase (CPY19) protein levels in testis of GPRC6A^{-/-} mice by Western Blot analysis (FIG. 3D) that was localized by immunohistochemistry (FIG. 3E) to Leydig cells, sertoli cells, and spermatocytes site where CPY19 is known to be expressed. Aromatase protein level was slightly increased by approximately 11% in testis of GPRC6A^{-/-} male mice (FIG. 3D). The significance of these changes in explaining the alterations in the testosterone and estradiol levels are uncertain, since we did not measure aromatase enzyme activity.

We found substantially no reductions in the expression of P450 17α-hydroxylase gene (Cyp17), an enzyme that converts pregnenolone to dehydroandrosterone (DHEA) in the testosterone synthesis pathway, or estrogen sulfotransferase gene (EST/Sult1e1), which catalyzes the sulfoconjugation and inactivation of estrogens, in the GPRC6A^{-/-} male mice testes by real-time PCR (FIGS. 3F and 3G).

Similarly, aromatase and Sultlel were not different in brain, fat, liver and pituitary of GPRC6A deficient and wild-type mice (data not shown). Gonadotrophin-releasing hormone gene (GnRH) message expression in the brain, however, was not altered in GPRC6A null mice (FIG. 3H-3I).

5.

For GPRC6A gene expression, the probe was amplified by RT-PCR using following intron spanning primer: mGPRC6A.189F (in Exon I) cgggatccagacgaccacaaatccag (SEQ ID NO: 25) and mGPRC6A.539R (spanned over Exon II and III) ccaagcttgattcataactcacctgtggc (SEQ ID NO: 26). After RT-PCR, the product was subclone into pre-cut by BamH I and Hind III of pBluescript SK(+). Using T7 promoter will create sense RNA, and T3 promoter will create Anti-sense RNA ribo-probe.

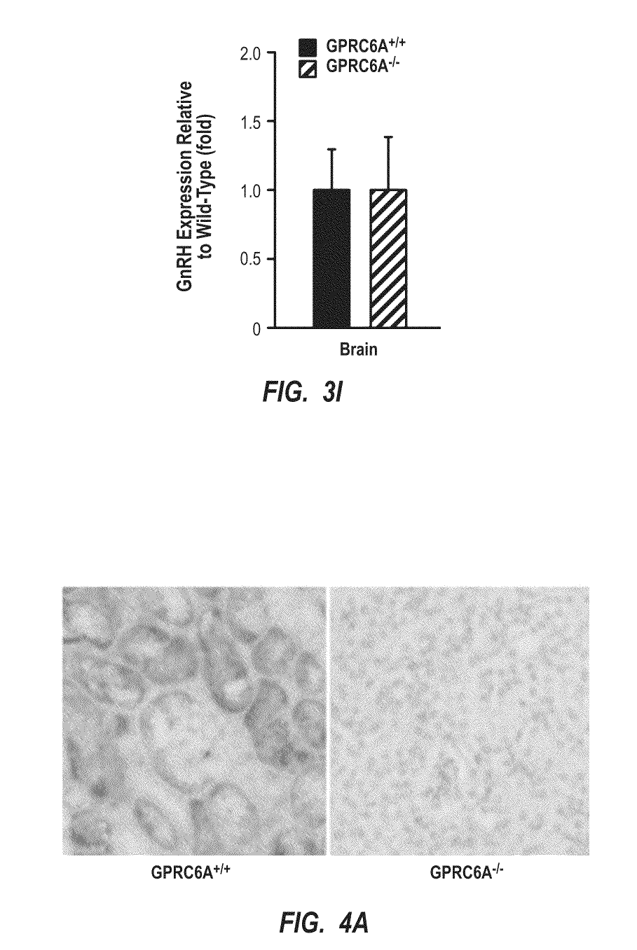

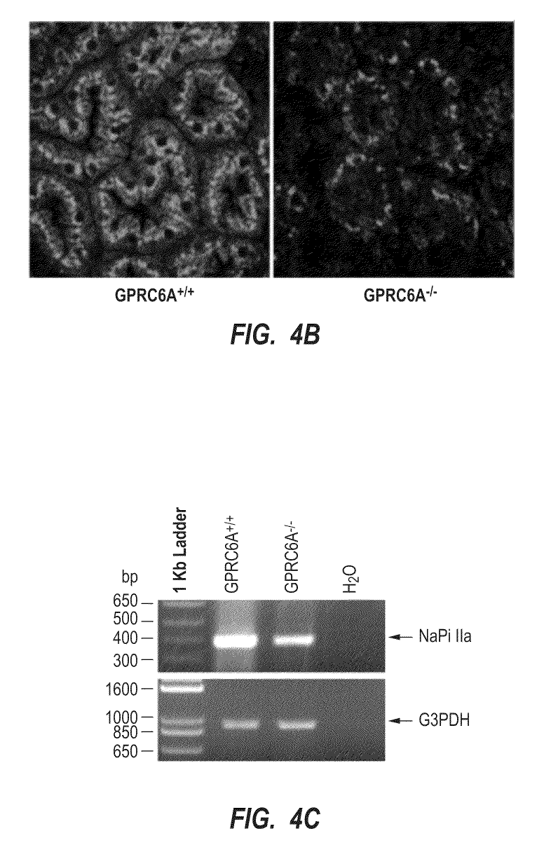

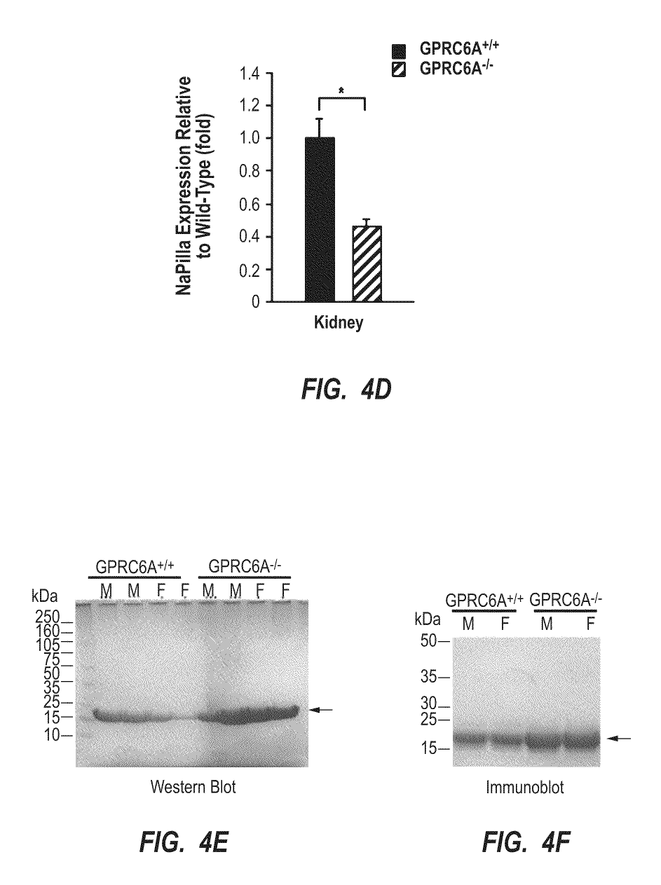

We previously demonstrated that GPRC6A is highly expressed in the kidney. We extended these observations by showing that GPRC6A is expressed in both proximal and distal tubules by in situ hybridization (FIG. 4A).

FIG. 4B shows the effect of GPRC6A ablation to reduce the transporter for phosphate in the proximal tubule of the kidney. Activation of GPRC6A might increase phosphate conservation whereas inhibition of GPRC6A would lead to increased phosphate excretion by the kidney

Interestingly, the expression of sodium-phosphate cotransporter, NaPi IIa, was decreased (both the transcript and protein) in GPRC6A^{-/-} mice (FIGS. 4C and D), suggesting adaptive responses in the kidney to excrete phosphate.

In addition, we found that urinary protein excretion was elevated in GPRC6A^{-/-} mice by Western blot analysis (FIG. 4E). Immunohistochemical analysis revealed that this protein band in the urine represents β2-microglobulin (FIG. 4F), providing evidence for abnormalities in the proximal tubule function in GPRC6A null mice.

6.

Serum was collected using a retroorbital bleeding technique. For urine samples collection, mice were placed in metabolic cages (Hatteras Instrument), and urine was collected for 24 h. The urine volume was measured before storage at −70° C. Serum testosterone and estradiol levels were measured by testosterone enzyme immunoassay test kit and estradiol (E2) enzyme immunoassay test kit from BioCheck, Inc. Follicle stimulating hormone (FSH) and luteinizing hormone (LH) were measured by mouse FSH radioimmunoassay and the mouse LH sandwich assay as described by the University of Virginia Center for Research in Reproduction Ligand and Analysis Core (NICHD (SCCPRR) Grant U54-HD28934). Serum and urinary calcium was measured by the colorimetric cresolphthalein binding method, and phosphorus was measured by the phosphomolybdate-ascorbic acid method. Serum TRAP was assayed with the ELISA-based SBA Sciences mouseTRAP™ assay. Serum PTH and 1,25(OH)₂ vitamin D were measured the kits from Immutopics, Inc. and Immunodiagnostic system, Ltd., respectively. Serum Fgf23 levels were measured by using FGF-23 ELISA kit (Kainos Laboratories Inc.) following the manufacturer's protocol. Creatinine was measured by the calorimetric alkaline picrate method (Sigma kit 555, Sigma-Aldrich). Urinary protein and Dpd were measured by Bio-Rad and Metra Biosystems, Inc., respectively.

We also found that GPRC6A^{-/-} mice had mild but significant increases in urinary calcium and phosphate excretion (calcium/creatinine ratio: 0.19±0.02; phosphorus/creatinine ratio: 5.32±0.31) compared to wild-type controls (calcium/creatinine ratio: 0.13±0.01; phosphorus/creatinine ratio: 3.93±0.28) (Table 1). The mild hypercalciuria was not evident at 6-weeks-of-age, but was present at subsequent ages, whereas the increased urinary phosphate levels were observed only in 16-week old GPRC6A^{-/-} mice. The level of serum phosphorus was also significantly higher in 16 week-old knockout mice (6.52±0.18 mg/dl) compared to wild-type littermates (5.18±0.21 mg/dl) (Table 1). Circulating concentrations of calcium, PTH, FGF23, and 1,25(OH)₂ vitamin D levels were not significantly different between wild-type and GPRC6A^{-/-} mice (Table 1).

Serum

| | Calcium | Phophorus | FGF23 | PTH | 1.25(OH)₂ Vit D₃ | |
|---|---|---|---|---|---|---|
| | (mg/dl) | (mg/dl) | (pg/ml) | (pg/ml) | (pmol/L) | TRAP(U/L) |
| GPRC6A^{+/+} | 6.05 ± 0.1 | 5.18 ± 0.21 | 74.51 ± 8.24 | 50.8 ± 7.96 | 366 17 ± 98.08 | 4.66 ± 0.55 |
| GPRC6A^{-/-} | 5.95 ± 0.11 | 6.52 ± 0.18** | 89.09 ± 10.71 | 50.26 ± 4.91 | 251.35 ± 38.58 | 5.25 ± 0.74 |

Urine

| | Dpd/Creatinine | Calcium/ | Phophorus/ | Protein/ | The |
|---|---|---|---|---|---|
| | Ratio | Creatinine Ratio | Creatinine Ratio | Creatinine Ratio | PhosphateExcretion |
| | (mg/mg) | (mg/mg) | (mg/mg) | (mg/mg) | Index |
| GPRC6A^{+/+} | 10.07 ± 1.29 | 0.16 ± 0.01 | 3.93 ± 0.28 | 15.32 ± 1.83 | 191.32 ± 46.28 |
| GPRC6A^{-/-} | 10.73 ± 1.39 | 0.19 ± 0.02* | 5.32 ± 0.31** | 22.65 ± 2.37* | 213.34 ± 35.58 |

Data are mean ± SEM. from more than 10 individual mice in each group.
*and **Significant difference from wild-type and GPRC6A null mice at p < 0.05 and p < 0.01 respectively.

In addition, we found that fasting serum glucose levels were significantly greater and insulin levels were lower in GPRC6A^{-/-} mice compared to wild-type littermates (Table 1) Additional figures, which are described below, shows abnormal glucose tolerance test and insulin tolerance test in GPRC6A null mice. Loss of GPRC6A leads to hyperglycemia and insulin resistance. Activation of GPRC6A would be predicted to lower glucose and increase insulin sensitivity, and be a therapy for Type II diabetes and metabolic syndrome.

7.

Wild-type and GPRC6A mouse kidney were routinely processed and embedded in paraffin. The paraffin sections at thickness of 5 μm were prepared and collected on commercially available, positively charged glass slides (Superfrost Plus, Fisher Scientific). The sections were dried on a hot plate to increase adherence to the slides. Representative sections were de-paraffined and re-hydrated through conventional methods. The sections were digested by 10 mg/ml hyaluronidase for 20 minutes. Nonspecific protein binding was blocked by incubation with 10% normal goat serum. The sections were incubated in polyclonal rabbit against mouse NaPi IIa (1:500 dilution) or polyclonal goat anti-human aromatase antibody (1:200 dilution) (CYP19, Santa Cruz Biotechnology, Inc.) at 4° C. overnight. The negative control sections were incubated with 0.01 M PBS. Thereafter, the sections were treated sequentially with FITC-conjugated Donkey anti Rabbit IgG secondary antibody (Jackson Labs). The nucleus was stained with ready to use Hoechst (Sigma).

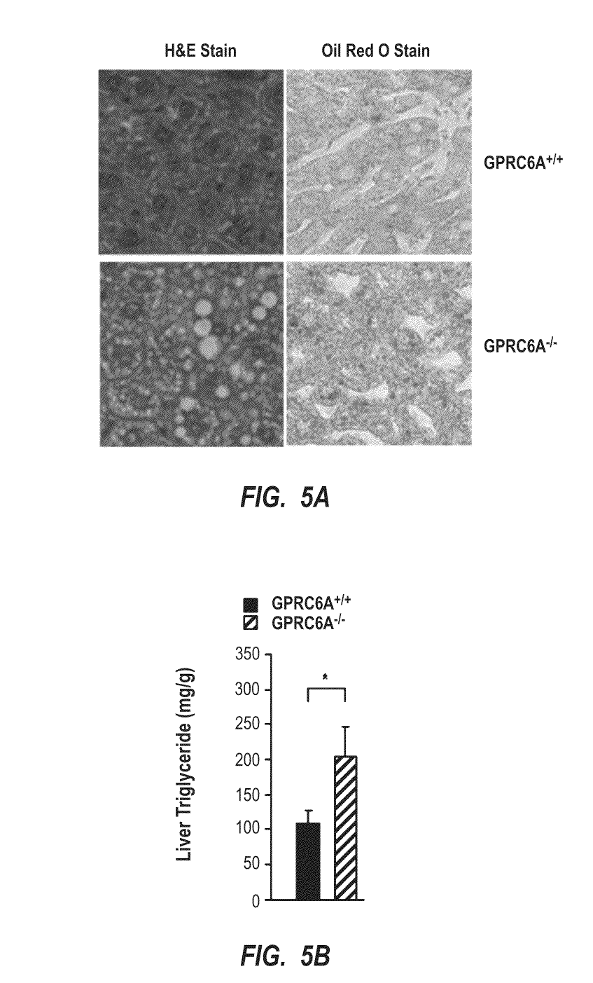

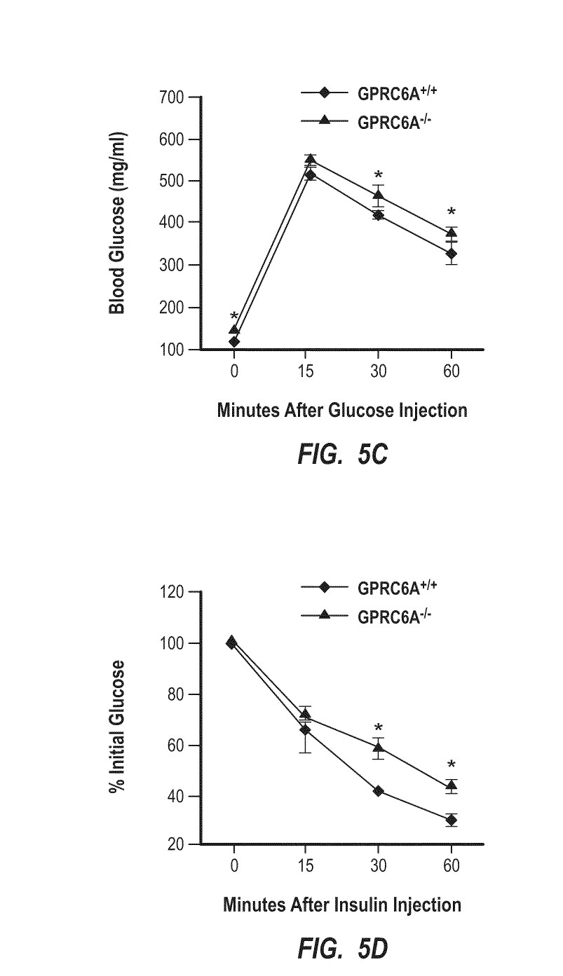

We also found that the liver of GPRC6A^{-/-} mice exhibited histological features of hepatic steatosis by H&E and Oil Red O staining (FIG. 5A). Lipid positive droplets were present in hepatocytes of GPRC6A^{-/-} mice but not wild-type mice. This correlated with increased triglyceride content in the livers of GPRC6A^{-/-} mice (FIG. 5B).

8.

For glucose tolerance test (GTT) glucose (2 g/kg body weight) was injected intraperitoneally (IP) after an overnight fast, and blood glucose was monitored using blood glucose strips and the Accu-Check glucometer (Roche) at indicated times. For insulin tolerance test (ITT) mice were fasted for 6 hours, injected IP with insulin (0.2 U/kg body weight, Lilly Research Laboratories), and blood glucose levels were measured at indicated times as described. ITT data are presented as percentage of initial blood glucose concentration.

Since fatty liver disease is a manifestation of “metabolic syndrome”, we examined GPRC6A^{-/-} mice for evidence of glucose intolerance. We performed glucose tolerance tests following IP injection of glucose (2 g/kg of body weight) after an overnight fast (GTT), and insulin tolerance tests by IP injection of insulin (0.2 units/kg of body weight) after 6 hours fast (ITT). These tests revealed that GPRC6A^{-/-} mice had a significantly higher serum glucose levels during the GTT and lower sensitivity to insulin than wild-type mice in the ITT (FIGS. 5C and 5D, respectively).

9.

Bone mineral density (BMD) of whole skeletons and femurs were assessed at 6, 8, 12, and 16 weeks of age using a PIXImus™ bone densitometer (Lunar Corp.) as previously described (Tu Q, Pi M, Karsenty G, Simpson L, Liu S, et al. (2003) Rescue of the skeletal phenotype in CasR-deficient mice by transfer onto the Gcm2 null background. J Clin Invest 111: 1029-1037).

Skeletons of mice were prelabeled twice with calcein (Sigma C-0875, 30 119/g body weight) by intraperitoneal injection at 8 and 3 days prior to sacrifice. Tibias and femurs were removed from 8- and 16-week-old mice, fixed in 70% ethanol, prestained in Villanueva stain and processed for methyl methacrylate embedding. Villanueva prestained sections were evaluated under fluorescent light.

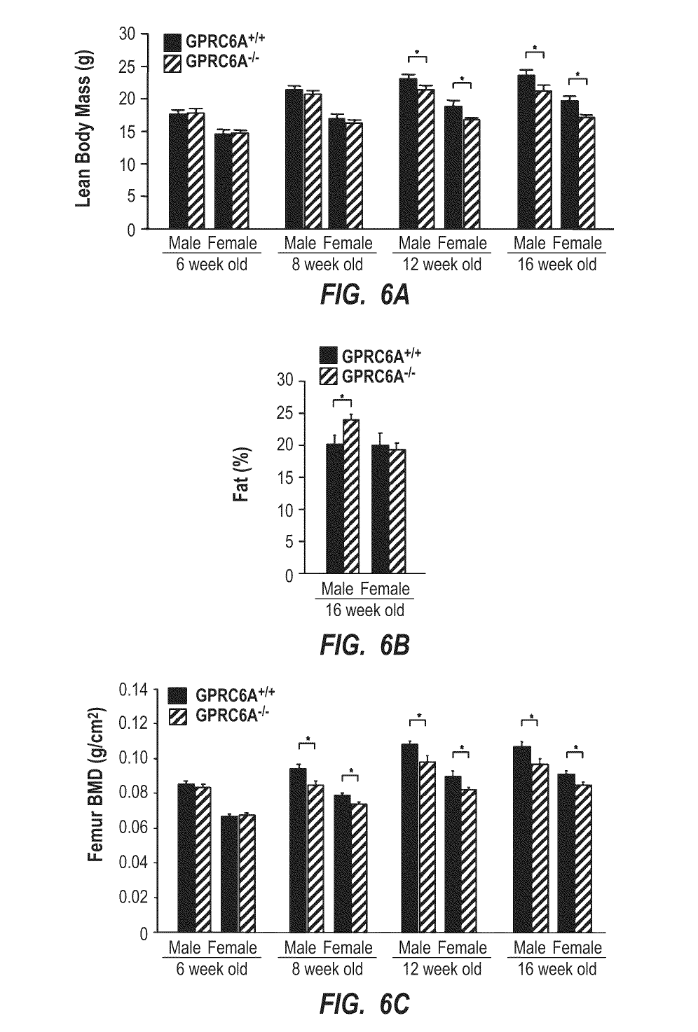

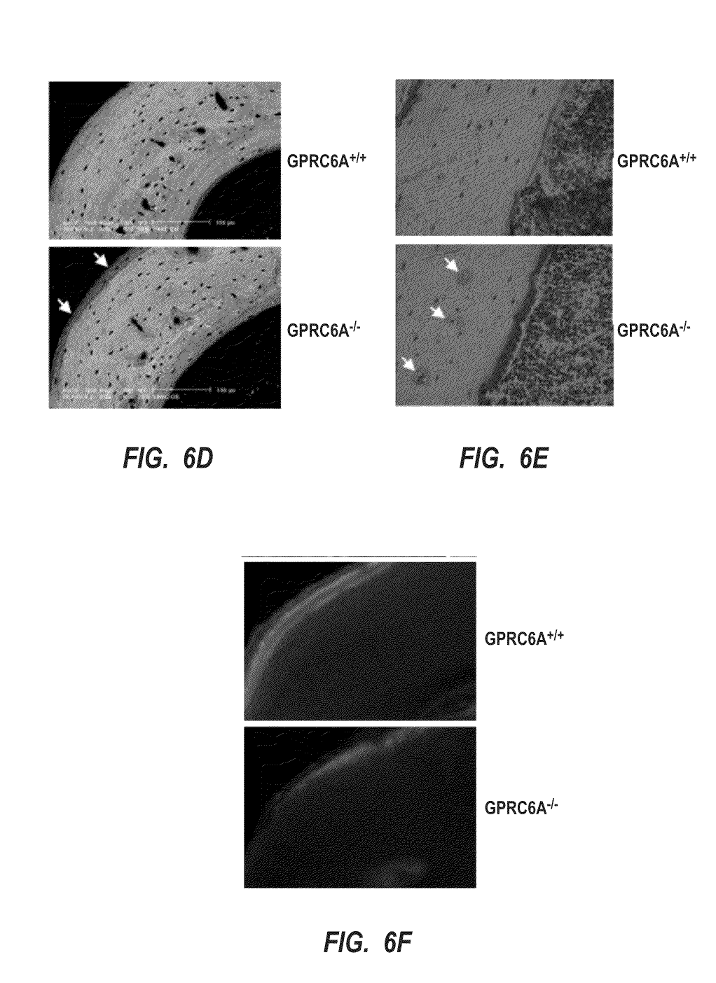

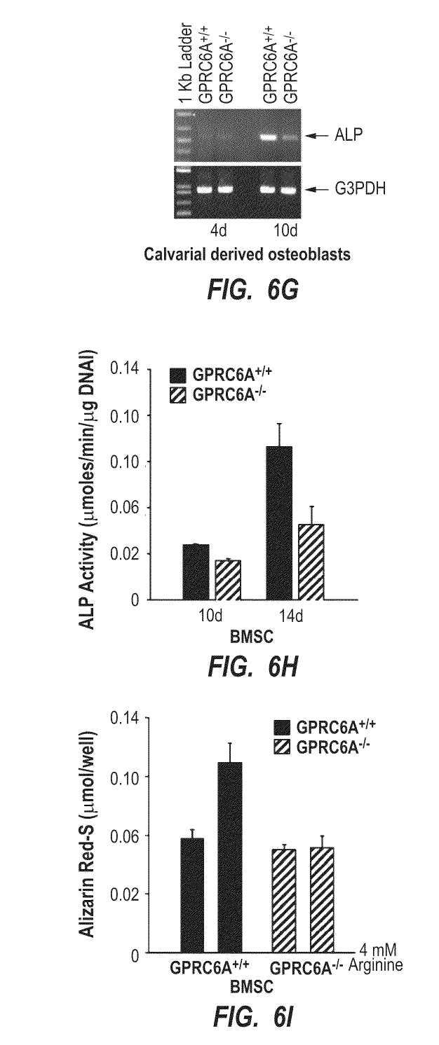

Both male and female GPRC6A^{-/-} mice had a significant reduction in lean body mass compared to wild-type littermates (7.9% and 10% in male and 11.2% and 13% in female GPRC6A^{-/-} mice at 12 and 16 weeks, respectively) as assessed by PIXImus™ densitometry (FIG. 6A). There were no apparent differences, however, in muscle histology between wild-type and GPRC6A^{-/-} mice (data not shown). Body fat as assessed by PIXImus™ densitometry (FIG. 6B) and white fat by gross inspection of various organs, such as testis, were increased in GPRC6A^{-/-} compared to wild-type mice. Both male and female GPRC6A^{-/-} mice did not have the expected age-dependent increase in bone mineral density (BMD). Indeed, BMD was significantly less at 8, 12, and 16 weeks-of-age in GPRC6A^{-/-} mice as compared to age-matched wild type mice (FIG. 6C).

To determine if the decreased bone density might be due to defective mineralization of bone, we performed backscatter EM and bone histological analysis. Briefly, plastic embedded bone from 8 week-old wild-type and GPRC6A knockout mice (GPRC6A^{-/-}) were cut and polished, and mounted on aluminum sockets with bone surface facing above, sputter-coated with gold and palladium, and examined with field emission scanning electron microscopy (Philips XL30, FEI Company) equipped with a backscatter electron imaging system. Backscatter EM of cortical bone demonstrated diminished mineralization surrounding osteocyte lacunae and on the perisoteal and endosteal surfaces (FIG. 6D).

Analysis of bone histology also revealed an increase in unmineralized osteoid surfaces (FIG. 6E) and diffuse calcien labeling of bone compared to the distinct double labels in wild-type mice (FIG. 6F), indicative of impaired mineralization, but no appreciable differences in osteoblasts or osteoclast number or appearance.

The distal femoral metaphyses were scanned using a micro-CT 40 (Scanco Medical AG); 167 slices of the metaphyses under the growth plate, constituting 1.0 mm in length, were selected. The three-dimensional (3D) images were generated using the following values for a gauss filter (sigma 0.8, support 1) and a threshold of 275. A 3D image analysis was performed to determine bone volume (BV/TV), trabecular number (Tb.N), trabecular thickness (Tb.Th), and trabecular separation (Tb.Sp). Cortical bone was measured on the mid-shaft region of cortical bone in 50 slices of the diaphysis, constituting 0.3 mm in length. The mean cortical thickness (Ct.Th) was determined at 8 different points on the cortical slice.

Although micro-CT analysis also detected a significant reduction in BOM in both the metaphyseal area, which predominately consists of trabecular bone, and the mid-shaft region, which is composed of cortical bone (Table 2), there were no demonstrable changes in bone structural parameters, including bone volume (BV/TV) and cortical thickness (Ct.Th), between GPRC6A^{-/-} and wild-type mice. This suggests that the decreased BMO might be due to defective mineralization of bone.

TABLE 2
μ-CT analysis of bone from 16 week-old wild-type and GPRC6A
knockout mice.

| | | | Bone |
|---|---|---|---|
| | | | Density |
| | BV/TV | Ct. Th (mm) | (mg HA/ccm) |
| GPRC6A^{-/-} | 0.3678 ± 0.0124 | 0.2515 ± 0.0104 | 1561.5 ± 6.66* |
| GPRC6A^{+/+} | 0.3723 ± 0.0112 | 0.255 ± 0.01 | 1618.9 ± 5.64 |

*Significant difference from wild-type and GPRC6A−/− mice at p < 0.05. Data represent the mean ± SEM from 6 mice for each group.

Assessment of expression of osteoblast markers in bone from 16-week-old GPRC6A^{-/-} mice, however, revealed reductions in osteocalcin, alkaline phosphatase, osteoprotegerin and Runx2-11 message levels compared to wild-type mice by real time RT-PCR (Table 3). The osteoclastic marker TRAP, chondrocyte marker Col II, and adipocyte markers aP2 and Lp1 were not significantly different between wildtype and GPRC6A^{-/-} mice (Table 3).

TABLE 3

| Gene | Accession Number | GPRC6A^{+/+} | GPRC6A^{-/-} |
|---|---|---|---|
| ALP | NM_007431 | 0.493 ± 0.096 | 0.194 ± 0.0045* |
| Osteocalcin | NM_007541 | 1.101 ± 0.068 | 0.411 ± 0.1* |
| Osteoprotegerin | MMU94331 | 0.0755 ± 0.021 | 0.0159 ± 0.0045* |
| Runx2-II | NM_009820 | 0.156 ± 0.034 | 0.0563 ± 0.0061* |
| Osterix | AF184902 | 0.00187 ± 0.00078 | 0.00136 ± 0.00047 |
| RANKL | NM_011613 | 0.000987 ± 0.00011 | 0.00124 ± 0.00037 |
| TRAP | NM_007388 | 0.793 ± 0.188 | 0.742 ± 0.12 |
| ColII | NM_031163 | 0.457 ± 0.219 | 0.186 ± 0.056 |
| aP2 | NM_024406 | 1.229 ± 0.305 | 1.624 ± 0.342 |
| LpI | NM_008509 | 0.0803 ± 0.0149 | 0.118 ± 0.054 |

*Denotes significant difference between wild-type and GPRC6A^{-/-}mice at p < 0.05. Data are mean ± S.E. from 8 weeks-old mice. Values are expressed relative to the housekeeping gene cyclaphilin A.
Abbreviations used are:
ALP. alkaline phosphatase;
aP2, adipocyte fatty acid-binding protein 2:
ColII, collagen type II, and
Lpl, lipoprotein lipase.

In addition, bone marrow stromal cells cultured from GPRC6A null mice exhibited reduced expression of alkaline phosphate expression and activity compared to wild-type cultures, which demonstrated the typical culture duration-dependent increase in alkaline phosphate (FIGS. 6G-6I). Reduced Alkaline phosphatase in primary calvarial osteoblasts and bone marrow stromal cells (BMSC) derived from GPRC6A^{-/-} mice. FIG. 6G shows alkaline phosphatase (ALP) expression that was measured by RT-PCR from 4- and 10-day primary osteoblasts cultures derived from 8-week GPRC6A^{+/+} and GPRC6A^{-/-} mouse calvaria. FIG. 6H shows alkaline phosphatase (ALP) activity in BMSCs from wild-type and GPRC6A^{-/-} mice cultured for 10 and 14 days. FIG. 6I shows alizarin Red-S for GPRC6A^{+/+} and GPRC6A^{-/-}. The alizarin Red-S stains mineralized matrix. The decrease in staining indicates that mineralization is impaired in the absence of GPRC6A. Thus, a GPRC6A antagonist can be used to inhibit mineralization of the extracellular matrix.

10.

HEK-293 cells were co-transfected with pcDNA3.mGPRC6A or pcDNA3 or pcDNA3.rCASR plasmid as previously described (Estrada, M., Uhlen, P. & Ehrlich, B. E. Ca2+ oscillations induced by testosterone enhance neurite outgrowth. Journal of cell science 119, 733-743 (2006). Agonist stimulation was performed in quiescent cells. Quiescence was achieved in subconfluent cultures by removing the media and washing with Hanks' Balanced Salt Solution (Invitrogen) to remove residual serum, followed by incubation for an additional 24 h in serum-free media. After agonist treatment at the specified concentrations and duration, cells were washed twice with ice-cold PSS and scraped into of lysis buffer (25 mM HEPES pH 7.2, 5 mM MgCl₂, 5 mM EDTA, 1% Triton X-100, 0.02 tablet/ml of protease inhibitor mixture). Equal amounts of lysates were subjected to 10% SDS-PAGE, and phospho-ERK1/2 levels were determined by immunoblotting using antiphospho-ERK1/2 mitogen-activated protein kinase antibody (Cell Signaling Technology). To confirm that variations in the amount of ERK did not contribute to stimulated ERK activity, we used an anti-ERK1/2 mitogen-activated protein kinase antibody (Cell Signaling Technology) to measure ERK levels. An anti-peptide antibody was raised in a rabbit against a peptide (AIHEKMLSSDDHPRRPQIQKC (SEQ ID NO: 27)) corresponding to a sequence in the extracellular domain of mouse GPRC6A (in exon 1 of mouse GPRC6A gene) produced by Abgent (San Diego, Calif.). For phospho-ERK, the phospho-ERK1/2 levels were determined by immunoblotting using anti-phospho-ERK1/2 mitogen-activated protein kinase antibody (Cell Signaling Technology). Urinary β2-microglobulin was detected by rabbit polyclonal anti-β2-microglobulin antibody (Abcam Inc.). For aromatase expression analysis, the rabbit polyclonal anti-aromatase antibody (Abcam Inc.) and goat anti-rabbit IgG HRP secondary antibody (Santa Cruz Biotechnology Inc.) were used. Mouse anti-Actin antibody (Santa Cruz Biotechnology Inc.) was used for control protein loading.

RT-PCR was also performed using two-step RNA PCR (Perkin-Elmer). In separate reactions, 2.0 μg of DNase-treated total RNA was reverse-transcribed into cDNA with the respective reverse primers specified below and Moloney murine leukemia virus reverse transcriptase (Life Technologies, Inc.). Reactions were carried out at 42° C. for 60 min followed by 94° C. for 5 min and 5° C. for 5 min. The products of first strand cDNA synthesis were directly amplified by PCR using AmpliTaq DNA polymerase (Perkin-Elmer). The primer sets used to amplify various gene transcripts with intron-spanning are as follows: hGPRC6A.F203: caggagtgtgttggctttga (SEQ ID NO: 30) and hGPRC6A.R630: atcaggtgagccattgcttt (SEQ ID NO: 31); mGPRC6A.189F: cgggatccagacgaccacaaatccag (SEQ ID NO: 32) and mGPRC6A.539R: ccaagcttgattcataactcacctgt (SEQ ID NO: 33); hAR.For1612: cctggcttccgcaacttacac (SEQ ID NO: 34) and hAR.Rev1779: ggacttgtgcatgcggtactca; G3PDH.F143: gaccccttcattgacctcaactaca (SEQ ID NO: 35); G3PDH.R1050: ggtcttactccttggaggccatgt (SEQ ID NO: 36).

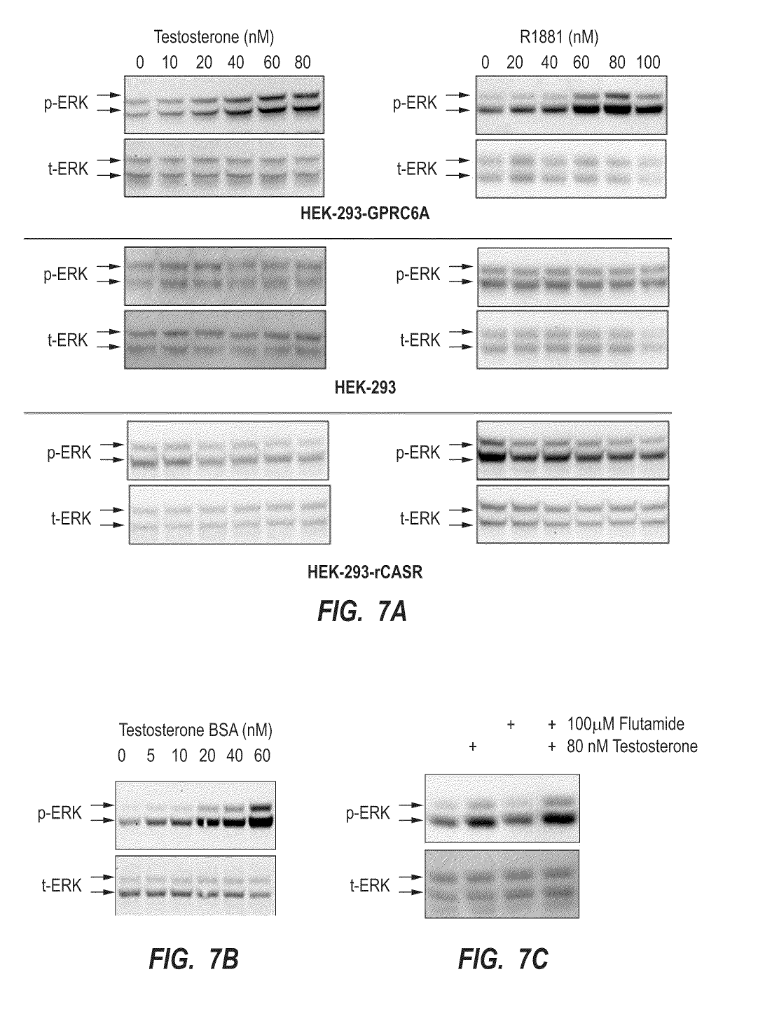

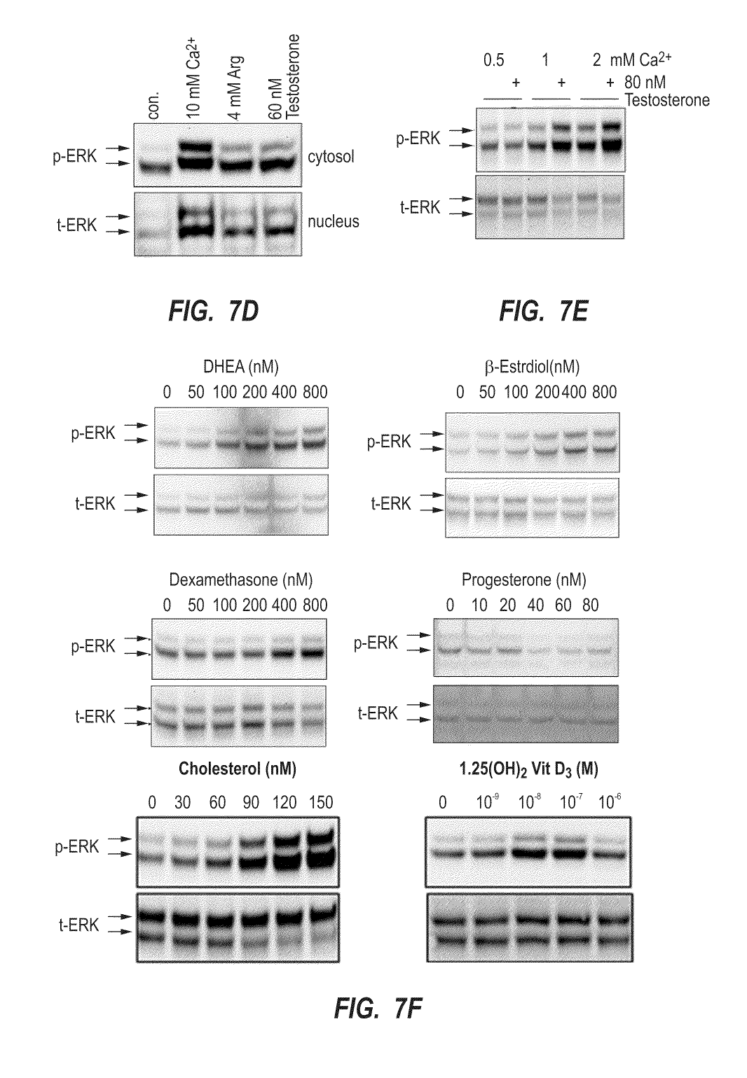

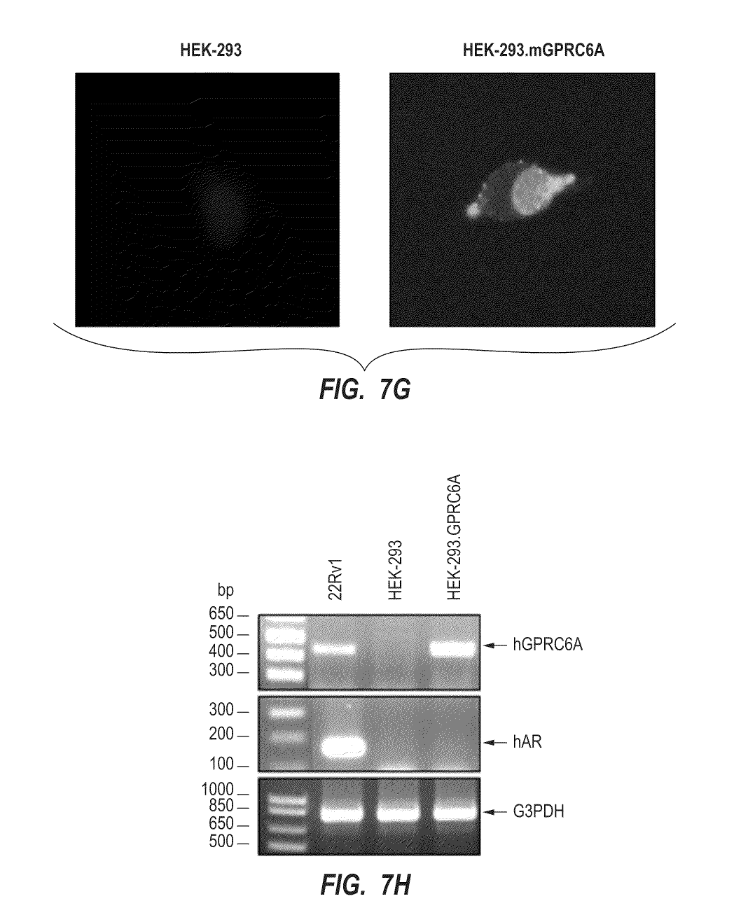

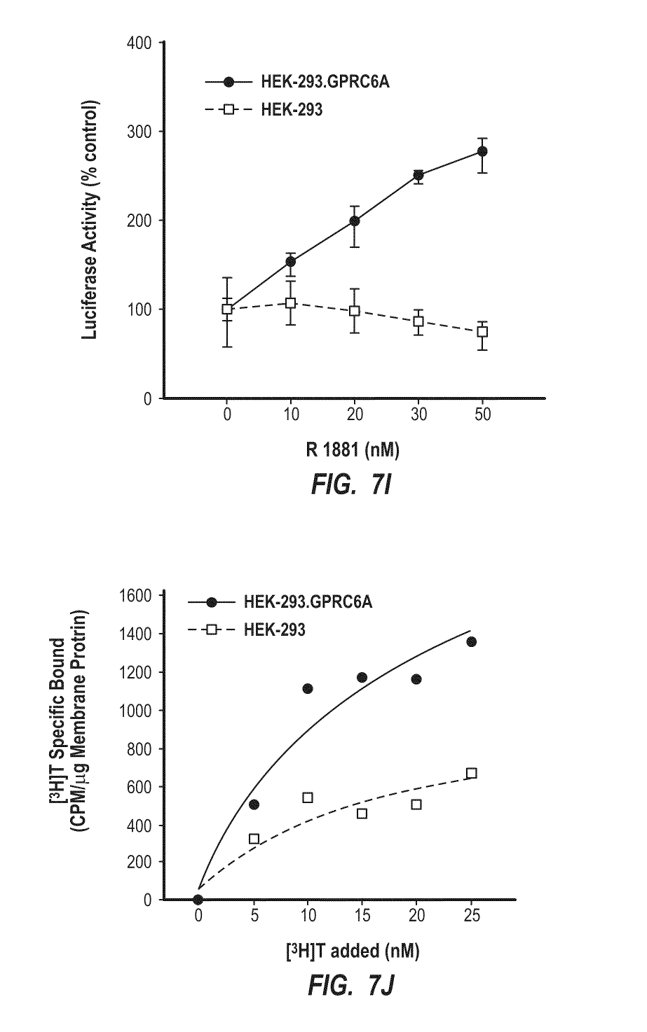

To define the role of GPRC6A in androgen-mediated cell function, we expressed GPRC6A in human embryonic kidney 293 cells (HEK-293) lacking the nuclear androgen receptor. First, we confirmed that HEK-293 cells express GPRC6A and classic androgen receptors by RT-PCR analysis. We used human prostate cancer cell line 22Rv-1 as positive control, and found that the GPRC6A and classic androgen receptor (AR) expressed in 22Rv-1 cells, but not expressed in HEK-293 cells (FIG. 7H). Therefore, we used HEK-293 as host cell to investigate the function of GPRC6A. In previously reports, GPRC6A is a amino acid- and calcium-sensing receptor. To explore the function of GPRC6A in respond to extracellular androgen, we first examined androgen response in HEK-293 cells cotransfected cDNAs of GPRC6A and a reporter gene construct, SRE-luciferase.

We found that testosterone and a synthetic androgen (R1881) stimulated extracellular signal-regulated kinase phosphorylation (phospho-ERK) in a dose-dependent fashion in HEK-293 cells transfected with GPRC6A, but not in the non-transfected HEK-293, HEK-293 transfected with GPRC6A (FIG. 7I), or HEK-293 transfected with the related G-protein-coupled calcium sensing receptor (CASR) (FIG. 7A). The concentration of testosterone required to activate GPRC6A was in the normal physiological range (e.g., 20 to 80 nM). Moreover, testosterone coupled to BSA, which is impermeable to the cell membrane, stimulated phospho-ERK in GPRC6A expressing HEK-293 cells (FIG. 7B), consistent with a cell surface effect. In contrast, the synthetic androgen receptor antagonist, flutamide, neither stimulated phospho-ERK nor inhibited GPRC6Adependent testosterone activation of phospho-ERK in HEK-293 cells (FIG. 7C). Testosterone also stimulated activation of phospho-ERK in both the cytosol and nucleus in GPRC6A expressing HEK-293 cells (FIG. 7D). Finally, testosterone activation of GPRC6A required medium calcium concentrations in excess of 0.5 mM (FIG. 7E), a concentration similar to the calcium requirement for amino acids and osteocalcin activation of GPRC6A.

Dehydroandrosterone (DHEA), 17p-estradiol, cholesterol, 1,25(OH)2Vit D3, and dexamethasone also stimulated the GPRC6A-mediated activation of phospho-ERK, but progesterone had no effect at concentrations up to 80 nM (FIG. 7F). Supraphysiological concentrations (60-80 nM) of 17p-estradiol were required to activate GPRC6A-mediated phosphorylation of ERK. ), As control, HEK-293 without GPRC6A did not responded (data not shown).

In addition, the synthetic androgen receptor antagonist, flutamide, neither stimulated phosphor-ERK nor inhibited GPRC6A-dependent testosterone activation of phospho-ERK in HEK-293 cells (FIG. 7C). In contrast, testosterone activation of GPRC6A required medium calcium concentrations in excess of 0.5 mM (FIG. 7E), a concentration similar to the calcium requirement for amino acids and osteocalcin activation of GPRC6A. The extracellular calcium may be also a positive modulator for the GPRC6A in response to steroids.

We previously demonstrated that GPRC6A overexpression results in cell surface expression of this receptor. To further confirm that GPRC6A is a membrane located G-protein coupled androgen sensing receptor, we used testosterone-BSA, testosterone coupled to BSA which is impermeable to the cell membrane; to stimulate the HEK-293 cells stably transfected GPRC6A. Testosterone-BSA induced a dose-dependent stimulation of phospho-ERK in GPRC6A expressing HEK-293 cells (FIG. 7B), consistent with a cell surface effect. Moreover, we elucidated androgen binding sites were identified on the surface of HEK-293 cells transfected with GPRC6A cDNA constructs. When cells were incubated with the impeded ligand testosterone-BSA coupled to FITC for 5 to 10 seconds, revealed increased fluorescence intensity on the surface of HEK-293 cells transfected with GPRC6A, but not in empty HEK-293 cells (FIG. 7G). In addition, we isolated the membrane fractions from the HEK-293 cells stably transfected GPRC6A and control HEK-293 cells. Significant amounts of specific testosterone binding were detected in plasma membranes of the HEK-293 cells transfected with GPRC6A, whereas negligible specific binding was detected in the plasma membranes of untransfected cells (FIG. 7J). All those data indicated that GPRC6A imparts cell surface binding of testosterone.

11.

Cell surface binding of testosterone was evaluated by modifications of previously described methods, Briefly, HEK-293 cells stably expressing GPRC6A or untransfected HEK-293 cells were grown on glass cover slips for 48 hours, washed with PSS and then incubated with testosterone-BSA-FITC at room temperature for 5 minutes, followed by two washings with PBS and cell fixation with 2% paraformaldehyde for 30 minutes. The cellular distribution of testosterone-BSA-FITC was then determined by fluorescent microscopy. FITC-conjugated testosterone accumulated on the surface of HEK-293 cells transfected with GPRC6A, but not in empty HEK-293 cells (FIG. 7G), indicating that GPRC6A imparts cell surface binding of testosterone.

12.

The femurs and tibias from 8-week-old wild-type and GPRC6A^{-/-} mice were dissected, the ends of the bones were cut, and marrow was flushed out with 2 mL of ice-cold a-MEM containing 10% FBS using a needle and syringe. A suspension of bone marrow cells was obtained by repeated aspiration of the cell preparation through a 22-gauge needle, and nucleated cells were counted with a hemocytometer. Cells were seeded into 6-well plates at a density of 3×10⁷ cells/mL and cultured for three days in a-MEM supplemented with 10% FBS, 100 kU/L of sodium penicillin G and 100 mg/L of streptomycin sulfate in a humidified incubator with 5% CO₂ and 95% air at a temperature of 37° C. On day 3, all nonadherent cells were then removed with the first medium change and then the adherent cells (representing bone marrow-derived mesenchymal stem cells, BMSCs) were grown for additional periods of up 3 days in the same medium. After overnight quiescence, the cells were stimulated for 5 minutes by testosterone and p-estradiol at the concentrations as indicated.

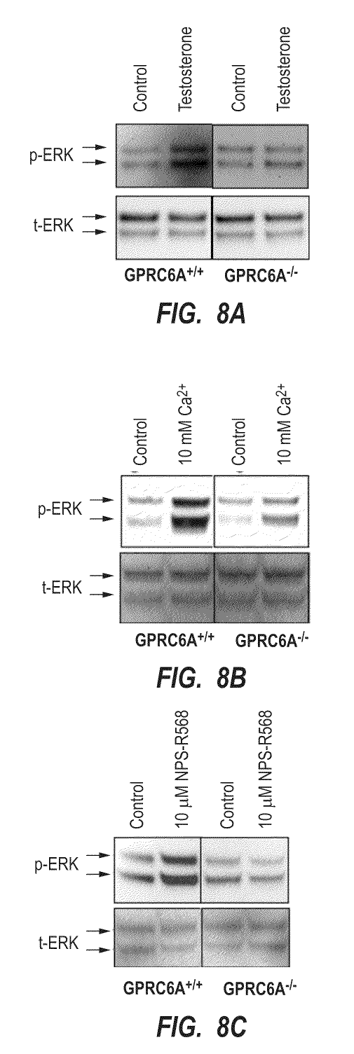

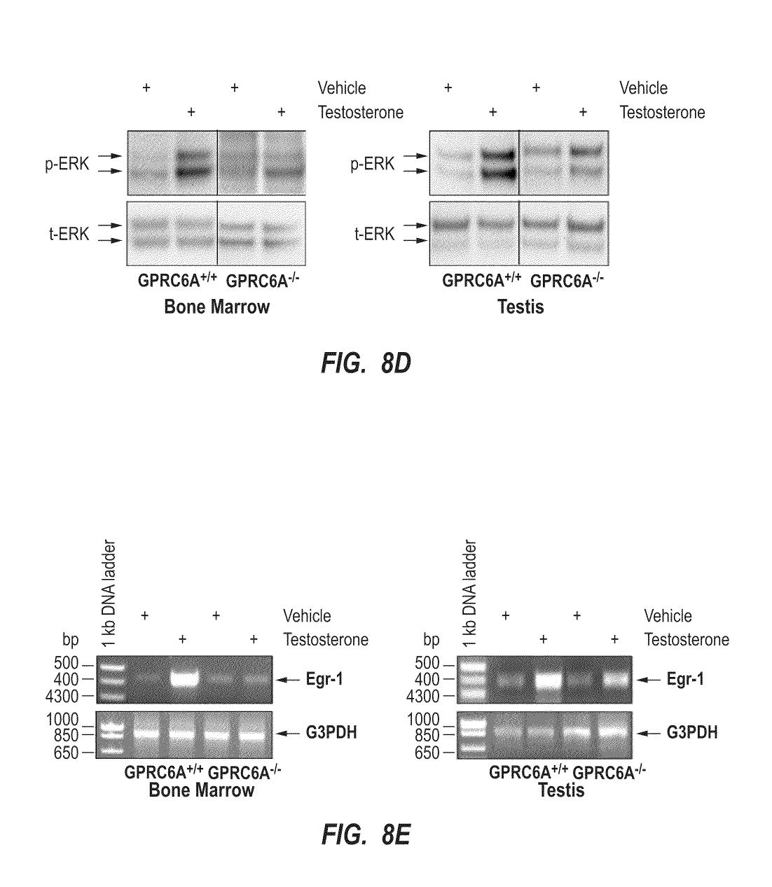

The non-genomic effects of androgens are present in many cell types, including osteoblasts and bone marrow stromal cells (BMSC) 1,10,18-20. Therefore, we next compared the ability of BMSC obtained from wild-type and GPRC6A^{-/-} mice to respond to testosterone added to the culture media (FIG. 8A). We observed that testosterone at concentrations up to 80 nM had only minimal effects to stimulate phospho-ERK activity in GPRC6A^{-/-} mice compared to its substantial stimulation of ERK in wild-type cells (FIG. 8A). BMSC derived from GPRC6A^{-/-} mice also displayed an attenuated response to extracellular calcium and calcimimetics (FIGS. 8B and 8C).

To establish a linkage between non-genomic effects of androgens and tissue responses in vivo, we examined the impact of loss of GPRC6A on the capacity of testosterone to stimulate phospho-ERK activity and early growth-responsive 1 (Egr-1) expression in bone marrow and testes in vivo (FIGS. 8D and 8E). To accomplish this, we administered testosterone at a dose of 200 MG/KG or vehicle intraperitonealy to wild-type and GPRC6A^{-/-} male mice. We found that testosterone treatment stimulated both phospho-ERK activity and Egr-1 expression in bone marrow and testes of wild-type mice, but this response was markedly attenuated in GPRC6A^{-/-} mice (FIGS. 8D and 8D).

In summary, we have shown that GPRC6A has multiple functions as evidenced by abnormalities in GPRC6A null mice that include alterations in circulating testosterone and estrogen levels and feminization of male mice, defects of bone density and bone cell function and abnormalities in the renal handling of calcium and phosphate, hyperglycemia and liver steatosis. The ligand profile of GPRC6A, which includes extracellular calcium, calcimimetics, amino acids, and osteocalcin, along with the complex phenotype of GPRC6A null mice suggests that GPRC6A is an anabolic receptor that responds to a variety of nutritional and hormonal signals and may serve to coordinate the functions of multiple organs in response to changes of these ligands. Thus, regulation of GPRC6A can be used in the treatment, inhibition, and prevention of diseases associated with a non-genomic androgen response. Increasing the activity or amount of GPRC6A can increase a non-genomic androgen response, and decreasing the activity or amount of GPRC6A can decrease a non-genomic androgen response.

13.

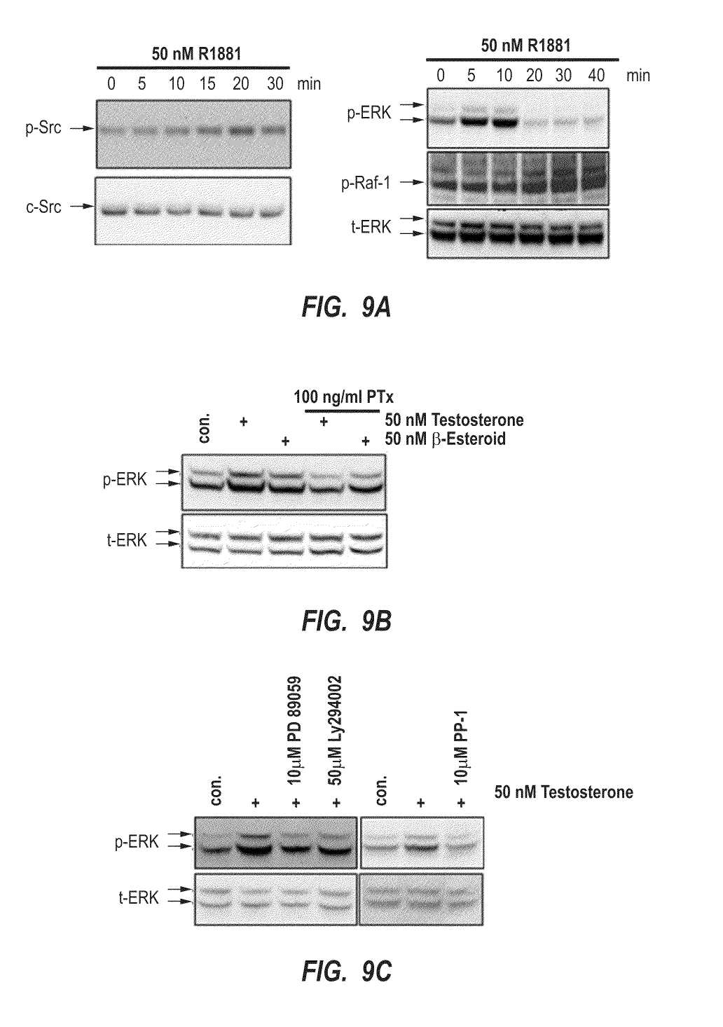

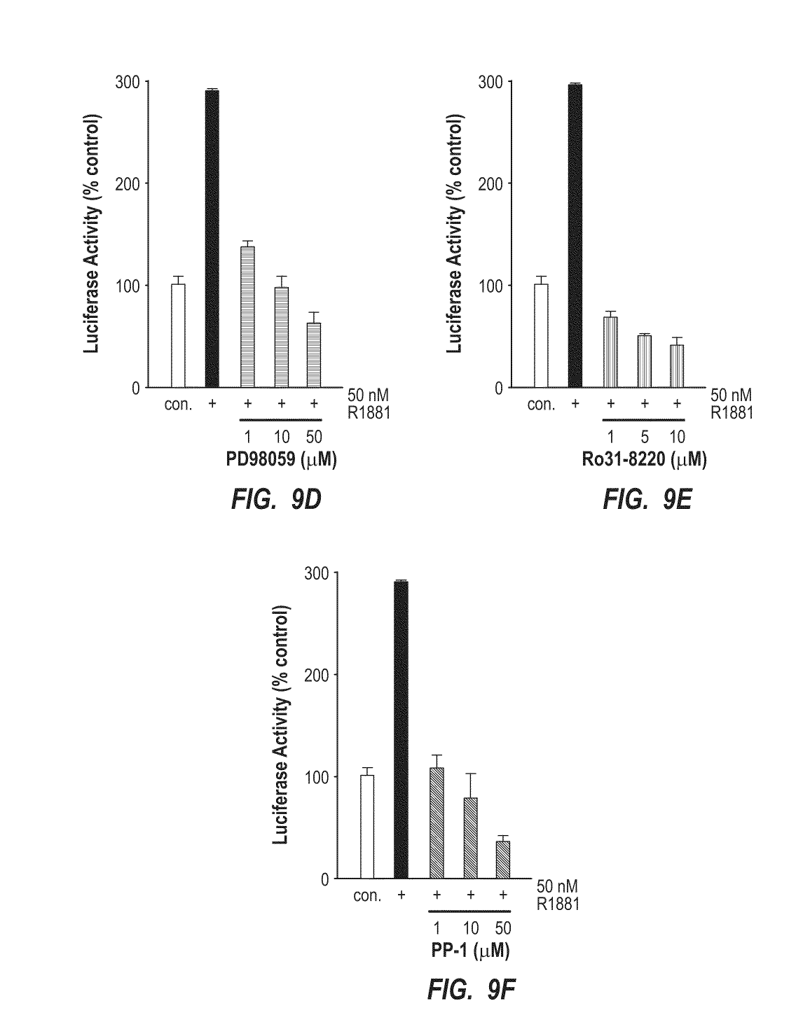

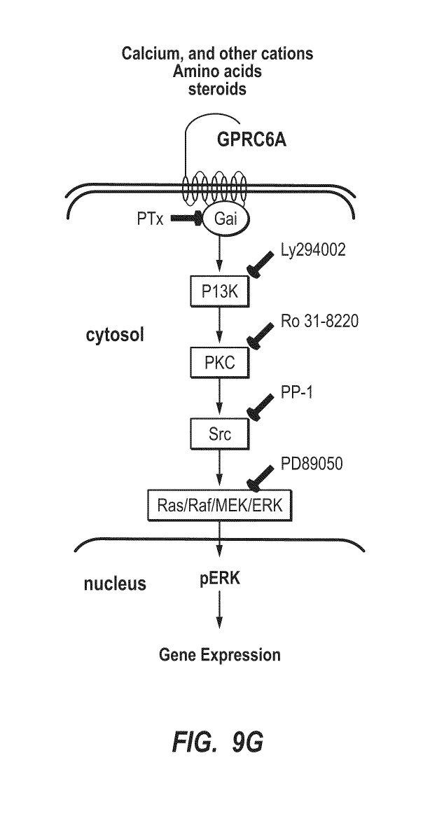

The non-genomic actions of androgens have been implicated in a number of cellular effects, resulting in stimulation of Src kinase activity within minutes in the LNCaP prostate cancer cell line in response to 10 nM R1881, and stimulation of Akt activity in the osteoblastic cells in response to 10 nM DHT. Several physiological processes arising as a result of the rapid action of the Src-Ras-ERK signaling pathway in steroids non-genomic action also have been reported. To define the role of GPRC6A in androgen-mediated cell signaling activation, we examine the Src and Raf-1 are involved in GPRC6A-mediated androgen stimulated intercellular signaling pathway. The results of western blot revealed a synthetic androgen, R1881, stimulated GPRC6A-mediated activation of phospho-Src (FIG. 9A) and phospho-Raf-1 (FIG. 9A).

HEK-293 cells were co-transfected with pcDNA3.mGPRC6A and SRE-luciferase reporter gene plasmid. Quiescence of transfected cells was achieved in subconfluent cultures by removing the media and washing with Hanks' balanced salt solution (Invitrogen) to remove residual serum followed by incubation for an additional 24 h in serum-free quiescent media. Luciferase activity was assessed after 6 h of stimulation. The luciferase activity in cell extracts was measured using the luciferase assay system (Promega) following the manufacturer's protocol using a BG-luminometer (Gem Biomedical, Inc., Hamden, Conn.).

Membranes from HEK and HEK stably transfected with GPRC6A were prepared and stored at −80° C. The membrane preparations were diluted to 0.15-0.5 mg protein/ml in binding buffer (in mM: 20 HEPES, 100 NaCl, 6 MgCl₂, 1 EDTA, and 1 EGTA) immediately before all binding assays. Total binding saturation curves were generated by incubating 250 μl of membrane preparation and 250 μl of [³H]Testoterone (Testosterone-[1,2,6,7-3H(N)]; 1 mCi; Sigma, Chemicals, St. Louis, Mo.), dissolved in binding buffer (in mM: 20 HEPES, 100 NaCl, 6 MgCl₂, 1 EDTA, and 1 EGTA) for final reaction concentrations ranging from 0.3 to 25 nM, for 40 min. After the 40-min incubation, the binding reactions were terminated by rapidly filtering 400 μl of the reaction over a presoaked Whatman, glass-fiber filter (pore size, 1 μm) to separate bound steroid from free steroid. The filter was immediately washed twice with 12.5 ml of wash buffer (PBS) and placed in a scintillation vial. Radioactivity was counted in a liquid scintillation counter (Beckman Instruments, Fullerton, Calif.). All steps of the binding assays were conducted at 4° C.

Testosterone also stimulated activation of phospho-ERK in both the cytosol and nucleus in GPRC6A expressing HEK-293 cells (FIG. 7A). We and others have previously shown that mGPRC6A can couple to two different signaling pathways, Gαq and Gαi. To determine whether GPRC6A mediates signaling through which G-protein subunits by androgen stimulation, we expressed GPRC6A in HEK-293 cells and then stimulated with extracellular testosterone and β-esteroid at concentration of 50 nM. Those stimulated activation of phospho-ERK were significantly blocked by 100 ng/ml pertussis toxin (PTx) (FIG. 9B). Pertussis toxin catalyzes the transfer of ADP-ribose from NAD to the guanine nucleotide-binding regulatory protein to specify inhibit Gαi subunit. We also shown that the GPRC6A-mediated extracellular testosterone stimulated signaling were blocked by PD89059 (MAPK inhibitor), Ly294002 (PI3K inhibitor), PP-1 (Src inhibitor) and Ro31-8220 (PKC inhibitor) using either phospho-ERK or SRE-luciferase as read-outs (FIGS. 9D-9F). These results suggest that Gαi, PI3K, PKC, Src and Ras/Raf/ERK may be involved testosterone stimulated GPRC6A-mediated signaling pathway (FIG. 9G).

14.

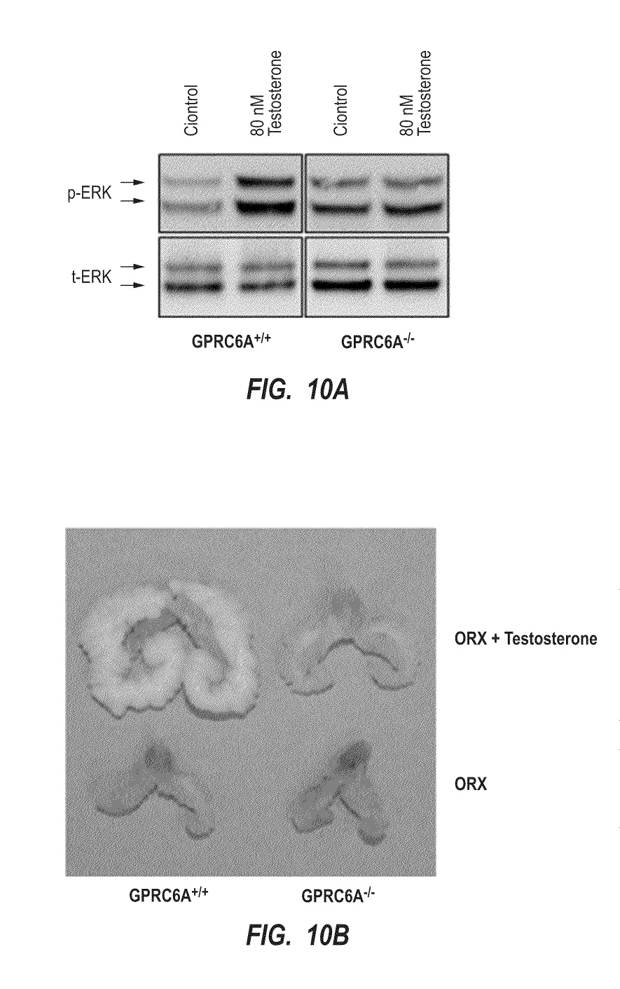

The non-genomic effects of androgens are present in many cell types, including osteoblasts and bone marrow stromal cells. Expression profiling by reverse transcriptase-mediated polymerase chain reaction (RT-PCR) revealed the presence of messenger RNA for GPRC6A in bone marrow, testis and seminal vesicle in wild-type mice, but not in GPRC6A null mice (GPRC6A^{-/-}) (FIG. 10A). We compared the ability of bone marrow stromal cells (BMSC) obtained from wild-type and GPRC6A^{-/-} mice to respond to testosterone added to the culture media (FIG. 10B). Indeed we observed that testosterone at concentrations up to 80 nM had only minimal effects to stimulate phospho-ERK activity in BMSC from GPRC6A^{-/-} mice compared to its substantial stimulation of ERK in cells from wild-type littermates (FIG. 10B). In addition, BMSC from GPRC6A^{-/-} mice failed to responds to extracellular calcium and the calcimimetic NPS-R568, whereas BMSC from WT mice exhibited both extracellular-calcium- and NPS-R568-dependent stimulation of ERK phosphorylation (data not shown).

Given that the testicular feminization phenotype is observed in androgen receptor mutant mice that exhibit reduced testosterone levels as well as end organ resistance to exogenous androgen administration, we examined if the GPRC6A^{-/-} mice exhibited resistance to the non-genomic effects of androgens. First, the male WT and GPRC6A^{-/-} littermates will be castrated by removal of the testicles (orchiectomy) and hormone recovered by implanting testosterone slow releasing pellet. The size of seminal vesicles from orchidectomized was shrunk, but not significantly different between wild-type and GPRC6A^{-/-} littermates (FIG. 8D). Then testosterone were given to orchidectomized GPRC6A^{-/-} and wild-type littermates, testosterone effectively recovered the size of seminal vesicles in wild-type mice, the recovery in GPRC6A^{-/-} mice was weaker compared to wild-type (FIG. 8D), which suggests that androgen actions mediated GPRC6A are impaired in GPRC6A^{-/-}mice.

15.

To establish a linkage between non-genomic effects of androgens and tissue responses in vivo, we examined the impact of loss of GPRC6A on the capacity of testosterone to stimulate phospho-ERK activity and early growth-responsive 1 (Egr-1) expression in bone marrow and testes (FIG. 8E). To accomplish this, we administered testosterone at a dose of 200 mg/kg or vehicle intraperitoneally to wild-type and GPRC6A^{-/-} male mice. Bone marrow was harvested at 20 and 60 minutes for assessment of ERK phosphorylation and Egr-1 mRNA expression. We found that testosterone treatment stimulated both phospho-ERK activity and Egr-1 expression in bone marrow and testes of wild-type mice, but this response was markedly attenuated in GPRC6A^{-/-} mice (FIG. 8E).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references or citations of publications or presentations (e.g., patents, published patent applications, journal articles, abstracts, posters, and the like) disclosed herein are incorporated into this provisional patent application by specific reference in their entirety.

What is claimed is:

1. A method for treating, inhibiting, or preventing a disorder, comprising:
identifying an individual with a disorder associated with GPRC6A; and
administering to the individual an agent capable of regulating an expression level and/or activity of GPRC6A.

2. The method of treating of claim 1, wherein said regulating increases or decreases the concentration of a sex hormone within said individual.

3. The method of treating of claim 1, wherein said regulating is upregulating said expression level and/or activity of said GPRC6A.

4. The method of treating of claim 3, wherein said upregulating is effected by administering to the individual an androgenergic agonist of said GPRC6A.

5. The method of treating of claim 3, wherein said disorder is an estrogen responsive breast cancer or ovarian cancer and said upregulating reduces the concentration of estradiol in the individual.

6. The method of treating of claim 3, wherein said disorder is osteoporosis or osteopenia and said upregulating increases bone density in said individual.

7. The method of treating of claim 3, wherein said disorder is an metabolic syndrome and said upregulating increases lean body mass and/or decreases body fat mass in the individual.

8. The method of treating of claim 3, wherein said disorder is diabetes.

9. The method of treating of claim 3, wherein said upregulating is effected by at least one approach selected from the group consisting of: (a) expressing in cells of said individual an exogenous polynucleotide encoding at least a functional portion of GPRC6A; (b) increasing expression of endogenous GPRC6A in said individual; (c) increasing endogenous GPRC6A activity in said individual; (d) introducing an exogenous polypeptide including at least a functional portion of GPRC6A to said individual; and (e) administering GPRC6A-expressing cells into said individual.

10. The method of treating of claim 1, wherein said regulating is down-regulating said expression level and/or activity of said GPRC6A.

11. The method of treating of claim 10, wherein said downregulating is effected by administering to said individual an androgenergic antagonist of said GPRC6A.

12. The method of treating of claim 10, wherein said disorder is prostate cancer.

13. The method of treating of claim 10, wherein said disorder is benign prostatic hypertrophy.

14. The method of treating of claim 10, wherein said downregulating is effected by introducing into said individual an agent selected from the group consisting of: (a) a molecule that binds said GPRC6A; (b) an enzyme which cleaves said GPRC6A; (c) an antisense polynucleotide capable of specifically hybridizing with at least part of an mRNA transcript encoding GPRC6A; (d) a ribozyme which specifically cleaves at least part of an mRNA transcript encoding GPRC6A; (e) a small interfering RNA (siRNA) molecule which specifically cleaves at least part of a transcript encoding GPRC6A; (f) a non-functional analogue of at least a catalytic or binding portion of said GPRC6A; and (g) a molecule which prevent GPRC6A activation or substrate binding.

15. A method for upregulating GPRC6A in a subject, comprising:
administering to the subject an androgenergic agonist of said GPRC6A in a therapeutically effective amount to upregulate GPRC6A.

16. A method as in claim 15, wherein the androgenergic agonist is selected from the group consisting of androgens, steroid hormones, androgenic hormones, anabolic steroids, testoids, testosterones, 19-carbon steroids, dehydroepiandrosterone (DHEA), dehydroepiandrosterone sulfate (DHEA-S), androstenedione, androstenediones, androstenediol, androsterone, dihydrotestosterone, androstanolone, fluoxymesterone, mesterolone, methyltestosterone, selective androgen receptor modulators (SARM), andarine, BMS-564,929, LGD-226, ostarine, S-40503, brimonidine tartrate, dexamethasone, indeloxazine hydrochloride, salts thereof, combinations thereof, and the like.

17. A method for downregulating GPRC6A in a subject, comprising:
administering to the subject an androgenergic antagonist of said GPRC6A in a therapeutically effective amount to downregulate GPRC6A.

18. A method as in claim 17, wherein the androgenergic antagonist is selected from the group consisting of allylestrenol, oxendolone, osaterone acetate, bicalutamide, steroidal anti-androgergic agents, medroxyprogesterone (MPA), cyproterone, cyproterone acetate (CPA), dienogest, flutamide, nilutamide, spironolactone, 5alpha-reductase inhibitors, dutasteride, finasteride, salts thereof, combinations thereof, and the like.

19. A GPRC6A knockout mouse comprising a GPRC6A gene having a deleted exon 2.

20. A mouse as in claim 19, wherein the mouse is heterozygous GPRC6A^{±}.

21. A mouse as in claim 19, wherein the mouse is homozygous GPRC6A^{-/-}.

22. A method for identifying a substance that modulates GPRC6A, said method comprising:
providing a cell expressing GPRC6A; and
screening the substance against the cell so as to determine whether or not the substance modulates GPRC6A.

23. A method as in claim 22, further comprising screening a library of substances.

24. A method as in claim 22, wherein the substance upregulates GPRC6A.

25. A method as in claim 22, wherein the substance downregulates GPRC6A.

26. A method as in claim 22, wherein the cell is transformed from a non-GPRC6A cell to a cell that expresses GPRC6A.
